(19) United States
(12) Patent Application Publication
(10) Pub. No.: US 2022/0117911 A1
(43) Pub. Date: Apr. 21, 2022

(54) METHODS AND COMPOSITIONS FOR MODULATING BLOOD-BRAIN BARRIER

(71) Applicants: INSERM (institut National de la Santé et de la Recherche Médicale), Paris (FR); Université de Paris, Paris (FR); Assistance Publique-Hôpitaux de Paris (APHP), Paris (FR)

(72) Inventors: Xavier DECLEVES, Paris (FR); Salvatore CISTERNINO, Paris (FR); Bruno SAUBAMEA, Paris (FR); Huilong LUO, Madison, WI (US)

(21) Appl. No.: 17/428,396

(22) PCT Filed: Feb. 3, 2020
(86) PCT No.: PCT/EP2020/052639

§ 371 Date: Aug. 4, 2021

(30) Foreign Application Priority Data
Feb. 4, 2019 (EP) 19305131.5

(51) Int. Cl. A61K 31/05; A61K 45/06

(52) U.S. Cl. CPC A61K 31/05; A61K 45/06

(57) ABSTRACT

The present invention relates to a method for modulating blood-brain barrier (BBB) in a subject comprising a step of administering said subject with a therapeutically effective amount of a modulator of transient receptor potential vanilloid-2 (TRPV2). For the first time, inventors have shown that TRPV2 is present in endothelial cells of BBB. More particularly, Inventor's results show that cannabidiol (CBD), at extracellular concentrations close to those observed in plasma of patients treated by CBD, and induces proliferation, migration, tubulogenesis and TEER increase in human brain endothelial cells, suggesting TRPV2 as a potent target for modulating the human BBB.

### FIELD OF THE INVENTION

The invention is in the field of neurology. More particularly, the invention relates to methods and composition for modulating blood-brain barrier.

### BACKGROUND OF THE INVENTION

The biochemical and functional features of brain microvessels endothelial cells, held together by tight junctions and forming the blood-brain barrier (BBB), regulate the molecular and cellular trafficking between blood and the brain parenchyma, thus maintaining the brain homeostasis milieu. BBB dysfunctions, as a cause or a consequence, are increasingly recognized in CNS disorders such as multiple sclerosis, epilepsy, neurodegenerative and psychiatric diseases¹ making it essential to define BBB drug targets.

The human genome encodes 27 distinct TRP channels grouped into six subfamilies (TRPA, TRPC, TRPM, TRPML, TRPP and TRPV). They are involved in diverse physiological and pathological processes such as regulation of blood blow, nociception, hormone secretion, immune response and modulation of barrier properties. TRP channels are sensitive to a variety of stimuli, including receptor stimulation, temperature, plant-derived compounds, environmental irritants, osmotic pressure, mechanical stress, pH, and voltage from the extracellular and intracellular milieu. Activation of TRP increases transmembrane flux of selected inorganic monovalent or divalent cations (e.g. Na⁺, K⁺, Ca²⁺, Mg²⁺)⁷. Whereas these ion currents could be involved in the resting potential and excitability of neurons as measured by patch clamp techniques, other non-excitable cells such as endothelial cells could exhibit different role for TRP functions.

Indeed, Ca²⁺ dynamics in brain microvessel endothelial cells is regarded as a major determinant of BBB properties' and the role of TRPVs on intracellular Ca²⁺ dynamics in brain microvessel endothelial cells has been demonstrated for TRPV1⁹ and more recently for TRPV4¹⁰ in human brain endothelial cells. Some drug candidates targeting TRPV1, 3 or 4 have even already entered clinical trials¹¹ with much less attention for targeting TRPV2.

The blood-brain barrier (BBB) is formed by the brain capillary endothelium and excludes from the brain about 100% of large-molecule neurotherapeutics and more than 98% of all small-molecule drugs. Overcoming the difficulty of delivering therapeutic agents to specific regions of the brain presents a major challenge to treatment of most brain disorders. Therapeutic molecules and antibodies that might otherwise be effective in diagnosis and therapy do not cross the BBB in adequate amounts.

Accordingly, there is a need to find new tools to increase or decrease the blood brain barrier permeability.

### SUMMARY OF THE INVENTION

The invention relates to a method for modulating blood-brain barrier (BBB) in a subject comprising a step of administering said subject with a therapeutically effective amount of a modulator of transient receptor potential vanilloid-2 (TRPV2). In particular, the invention is defined by claims.

### DETAILED DESCRIPTION OF THE INVENTION

TRPV2 expression and its role on Ca2+ cellular dynamics, trans-endothelial electrical resistance (TEER), cell viability and growth, migration and tubulogenesis was evaluated in human primary cultures of BMEC (hPBMEC) or in the human cerebral microvessel endothelial hCMEC/D3 cell line. Abundant TRPV2 expression was measured in hCMEC/D3 and hPBMEC by qRT-PCR, Western blotting, non-targeted proteomics and cellular immunofluorescence studies. Intracellular Ca2+ levels were increased by heat and CBD, and blocked by the non-specific TRP antagonist ruthenium red (RR) and the selective TRPV2 inhibitor tranilast (TNL) or by silencing cells with TRPV2 siRNA. CBD dose-dependently induced hCMEC/D3 cell growth (EC50 0.3±0.1 this effect being fully abolished by TNL or TRPV2 siRNA. Wound healing assay showed that CBD induced cell migration which was also inhibited by TNL or TRPV2 siRNA. Tubulogenesis of hCMEC/D3 cells in 3D matrigel cultures was significantly increased by 41% and 73% after 7 h or 24 h CBD treatment, respectively, and abolished by TNL. CBD also increased TEER of hPBMEC monolayers cultured in transwell and this was blocked by TNL. Inventor's results show that CBD, at extracellular concentrations close to those observed in plasma of patients treated by CBD, induces proliferation, migration, tubulogenesis and TEER increase in human brain endothelial cells, suggesting TRPV2 as a potent target for modulating the human BBB.

Methods for Modulating Blood-Brain Barrier:

Accordingly, in a first aspect, the invention relates to a method for modulating blood-brain barrier (BBB) in a subject comprising a step of administering said subject with a therapeutically effective amount of a modulator of transient receptor potential vanilloid-2 (TRPV2).

In a particular embodiment, the invention relates to a method for treating a subject in need thereof comprising a step of administering said subject with a therapeutically effective amount of a modulator of TRPV2.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “blood brain barrier (BBB)” refers to a semipermeable border that separates the circulating blood from the brain and extracellular fluid in the central nervous system (CNS). The blood-brain barrier provides a defence against disease-causing pathogens and toxins that may be present in the blood. The biochemical and functional features of brain microvessels endothelial cells, held together by tight junctions and forming the blood-brain barrier (BBB), regulate the molecular and cellular trafficking between blood and the brain parenchyma, thus maintaining the brain homeostasis milieu.

The BBB, which is formed by brain endothelial cells, allows the passage of water, some gases, and lipid-soluble molecules by passive diffusion, as well as the selective transport of molecules such as glucose and amino acids that are crucial to neural function, while restricting the diffusion of microscopic objects (e.g., bacteria or cells such as leukocytes) and large or hydrophilic molecules into the cerebrospinal fluid (CSF).

As used herein, the term “modulating BBB” refers to stimulating or inhibiting cells proliferation, differentiation, or both proliferation and differentiation in the BBB. In the context of the invention, modulating refers to increasing or decreasing the permeability of BBB.

In a particular embodiment, the invention relates to a method for increasing blood brain barrier permeability in a subject comprising a step of administering said subject with a therapeutically effective amount of a modulator of transient receptor potential vanilloid-2 (TRPV2).

As used herein, the term “increasing the permeability of the BBB” refers to increase the permeability of BBB. Typically, the method according to the invention allows BBB to be more permeable for the treatments, for example increasing the amount or size of molecules or microscopic objects transported across the BBB. The method according to the invention is suitable to increase the permeability of the BBB of a subject to a molecule present in the blood stream of the subject.

In a particular embodiment, the invention relates to a method for decreasing blood brain barrier permeability in a subject comprising a step of administering said subject with a therapeutically effective amount of a modulator of transient receptor potential vanilloid-2 (TRPV2).

As used herein, the term “decreasing blood brain barrier permeability” refers to decrease the permeability of BBB. More particularly, when more the BBB is compromised allowing for the passage of larger and hydrophilic substances. Typically, the method according to the invention allows to inhibit the penetration of some microscopic objects (e.g., bacteria or cells such as leukocytes) and large or hydrophilic molecules into the cerebrospinal fluid (CSF). The term “decreasing blood brain barrier permeability” refers to decreasing the amount or size of molecules or microsopic objects transported across the BBB.

In this context, the method of the invention is suitable to treat a neuroinflammation, traumatic brain injury or ischemic stroke.

As part of the neurovascular unit, the blood-brain barrier (BBB) is a unique, dynamic regulatory boundary that limits and regulates the exchange of molecules, ions, and cells between the blood and the central nervous system. Disruption of the BBB plays an important role in the development of neurological dysfunction in ischemic stroke, traumatic brain injury or neuroinflammation.

As used herein, the term “ischemic stroke” is well-known in the art and refers to a blood clot that blocks or plugs a blood vessel in the brain.

As used herein, the term “traumatic brain injury (TBI)” is well-known in the art and refers to sudden damage to the brain caused by a blow or jolt to the head. Following stroke or TBI, there is loss of BBB tight junction integrity, leading to increased paracellular permeability, which results in vasogenic edema, hemorrhagic transformation, and increased mortality.

As used herein, the term “neuroinflammation” is well-known in the art and refers to the inflammation of the nervous tissue. The central nervous system (CNS) is typically an immunologically privileged site because peripheral immune cells are generally blocked by the BBB.

The methods and compositions as described herein can increase drug delivery to the brain. For example, the drug to be delivered to the brain can be a drug suitable for treating a brain pathology. For example, the methods and compositions as described herein can improve known methods of treatment for a brain pathology by allowing a drug or a therapeutic agent to reach the brain parenchyma by opening up the blood brain barrier. A brain pathology that can be treated with the disclosed compositions and methods can be a disease, disorder, or condition of the brain, such as brain cancer, a brain tumor, or any other neurological disorder, disease, or condition.

In a particular embodiment, the methods and compositions as described herein are suitable to repair the BBB. Typically, the brain is considered leaky when the blood-brain barrier has been compromised in some way. When the tight junctions become lost or broken, the BBB becomes more permeable and harmful substances can leak in. Harmful chemicals and proteins can damage the brain leading to inflammation; in other words, a leaky brain is an inflamed brain.

Accordingly, the invention relates also to a method for repairing the blood-brain barrier in a subject in need thereof comprising a step of administering to said subject a therapeutically effective amount of a modulator of transient receptor potential vanilloid-2 (TRPV2).

As used herein, the term “subject” refers to any mammals, such as a rodent, a feline, a canine, and a primate. In a particular embodiment, the subject is human. Particularly, in the present invention, the subject has or is susceptible to have a disorder selected from psychiatric/behavioral disorders and CNS diseases; encephalitis of the central nervous system, Parkinson's disease, epilepsy, neurological manifestations of HIV-AIDS, neurological sequela of lupus, Huntington's disease, and brain tumors. meningitis, multiple sclerosis, neuromyelitis optica, herpes simplex virus (HSV) encephalitis, and progressive multifocal leukoencephalopathy, schizophrenia, manic depression, dementia, and bipolar disorder. In a particular embodiment, the subject has or is susceptible to have a BBA altered or the brain is leaky. Typically the subject has or is susceptible to have neuro-inflammatory disease.

In a particular embodiment, the present invention provides methods and compositions for use in the treatment of ischemic stroke, traumatic brain injury, or neuroinflammation.

In a particular embodiment, the present invention provides methods and compositions for use in the treatment of brain tumors, brain cancer, or spinal cord tumors. For example, a brain or spinal cord tumor that can be treated with the methods and compositions as described herein can be Acoustic Neuroma; Astrocytoma (e.g., Grade I—Pilocytic Astrocytoma, Grade II—Low-grade Astrocytoma, Grade III—Anaplastic Astrocytoma, Grade IV—Glioblastoma (GBM), a juvenile pilocytic astrocytoma); Atypical Teratoid Rhaboid Tumor (ATRT); Chordoma; Chondrosarcoma; Choroid Plexus; CNS Lymphoma; Craniopharyngioma; cysts; Ependymoma; Ganglioglioma; Germ Cell Tumor; Glioblastoma (GBM); Gliomas (e.g., Brain Stem Glioma, Ependymoma, Mixed Glioma, Optic Nerve Glioma, Subependymoma); Hemangioma; Lipoma; Lymphoma; Medulloblastoma; Meningioma; Metastatic Brain Tumors; Neurofibroma; Neuronal & Mixed Neuronal-Glial Tumors; Non-Hodgkin lymphoma; Oligoastrocytoma; Oligodendroglioma; Pineal Tumors; Pituitary Tumors; Primitive Neuroectodermal (PNET); Other Brain-Related Conditions; Schwannoma (neurilemmomas); Brain Stem Glioma; Craniopharyngioma; Ependymoma; Juvenile Pilocytic Astrocytoma (JPA); Medulloblastoma; Optic Nerve Glioma; Pineal Tumor, Primitive Neuroectodermal Tumors (PNET); or Rhabdoid Tumor. In a particular embodiment, the brain tumor is glioblastoma and gliomas.

In another embodiment, the method according to the invention, wherein the subject has or is susceptible to have neurological diseases, disorders, or conditions. For example, a neurological disease, disorder, or condition can be treated with the methods and compositions as described herein. The method according to the invention, wherein the subject has or is susceptible to have Abulia; Agraphia; Alcoholism; Alexia; Alien hand syndrome; Allan-Hemdon-Dudley syndrome; Alternating hemiplegia of childhood; Alzheimer's disease; Amaurosis fugax; Amnesia; Amyotrophic lateral sclerosis (ALS); Aneurysm; Angelman syndrome; Anosognosia; Aphasia; Apraxia; Arachnoiditis; Amold-Chiari malformation; Asomatognosia; Asperger syndrome; Ataxia; Attention deficit hyperactivity disorder; ATR-16 syndrome; Auditory processing disorder; Autism spectrum; Behcets disease; Bipolar disorder; Bell's palsy; Brachial plexus injury; Brain damage; Brain injury; Brain tumor; Brody myopathy; Canavan disease; Capgras delusion; Carpal tunnel syndrome: Causalgia; Central pain syndrome; Central pontine myelinolysis; Centronuclear myopathy; Cephalic disorder; Cerebral aneurysm; Cerebral arteriosclerosis; Cerebral atrophy; Cerebral autosomal dominant arteriopathy with subcortical infarcts and leukoencephalopathy (CADASIL); Cerebral dysgenesis-neuropathy-ichthyosis-keratoderma syndrome (CEDNIK syndrome); Cerebral gigantism; Cerebral palsy; Cerebral vasculitis; Cervical spinal stenosis; Charcot-Marie-Tooth disease; Chiari malformation; Chorea; Chronic fatigue syndrome: Chronic inflammatory demyelinating polyneuropathy (CIDP); Chronic pain; Cockayne syndrome; Coffin-Lowry syndrome; Coma; Complex regional pain syndrome; Compression neuropathy; Congenital facial diplegia; Corticobasal degeneration; Cranial arteritis; Craniosynostosis; Creutzfeldt-Jakob disease; Cumulative trauma disorders; Cushing's syndrome; Cyclothymic disorder; Cyclic Vomiting Syndrome (CVS); Cytomegalic inclusion body disease (CIBD); Cytomegalovirus Infection; Dandy-Walker syndrome; Dawson disease; De Morsier's syndrome; Dejerine-Klumpke palsy; Dejerine-Sottas disease; Delayed sleep phase syndrome; Dementia; Dermatomyositis; Developmental coordination disorder; Diabetic neuropathy; Diffuse sclerosis; Diplopia; Disorders of consciousness; Down syndrome; Dravet syndrome; Duchenne muscular dystrophy; Dysarthria; Dysautonomia; Dyscalculia; Dysgraphia; Dyskinesia; Dyslexia; Dystonia; Empty sella syndrome; Encephalitis; Encephalocele; Encephalotrigeminal angiomatosis; Encopresis; Enuresis; Epilepsy; Epilepsy-intellectual disability in females; Erb's palsy; Erythromelalgia; Essential tremor; Exploding head syndrome; Fabry's disease; Fahr's syndrome; Fainting; Familial spastic paralysis; Febrile seizures; Fisher syndrome; Friedreich's ataxia; Fibromyalgia; Foville's syndrome; Fetal alcohol syndrome; Fragile X syndrome; Fragile X-associated tremor/ataxia syndrome (FXTAS); Gaucher's disease; Generalized epilepsy with febrile seizures plus; Gerstmann's syndrome; Giant cell arteritis; Giant cell inclusion disease; Globoid Cell Leukodystrophy; Gray matter heterotopia; Guillain-Barre syndrome; Generalized anxiety disorder; HTLV-1 associated myelopathy; Hallervorden-Spatz syndrome; Head injury; Headache; Hemifacial Spasm; Hereditary Spastic Paraplegia; Heredopathia atactica polyneuritiformis; Herpes zoster oticus; Herpes zoster Hirayama syndrome; Hirschsprung's disease; Holmes-Adie syndrome; Holoprosencephaly; Huntington's disease; Hydranencephaly; Hydrocephalus; Hypercortisolism; Hypoxia; Immune-Mediated encephalomyelitis; Inclusion body myositis; Incontinentia pigmenti; Infantile Refsum disease; Infantile spasms; Inflammatory myopathy; Intracranial cyst; Intracranial hypertension; Ischemic stroke; Isodicentric 15; Joubert syndrome; Karak syndrome; Kearns-Sayre syndrome; Kinsboume syndrome; Kleine-Levin syndrome; Klippel Feil syndrome; Krabbe disease; Kufor-Rakeb syndrome; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Leukoencephalopathy with vanishing white matter; Lewy body dementia; Lissencephaly; Locked-in syndrome; Lou Gehrig's disease (e.g., amyotrophic lateral sclerosis); Lumbar disc disease; Lumbar spinal stenosis; Lyme disease—Neurological Sequelae; Machado-Joseph disease (Spinocerebellar ataxia type 3); Macrencephaly; Macropsia; Mal de debarquement; Megalencephalic leukoencephalopathy with subcortical cysts; Megalencephaly; Melkersson-Rosenthal syndrome; Menieres disease; Meningitis; Menkes disease; Metachromatic leukodystrophy; Microcephaly; Micropsia; Migraine; Miller Fisher syndrome; Mini-stroke (transient ischemic attack); Misophonia; Mitochondrial myopathy; Mobius syndrome; Monomelic amyotrophy; Morvan syndrome; Motor Neurone Disease (e.g., amyotrophic lateral sclerosis); Motor skills disorder; Moyamoya disease; Mucopolysaccharidoses; Multi-infarct dementia; Multifocal motor neuropathy; Multiple sclerosis; Multiple system atrophy; Muscular dystrophy; Myalgic encephalomyelitis; Myasthenia gravis; Myelinoclastic diffuse sclerosis; Myoclonic Encephalopathy of infants; Myoclonus; Myopathy; Myotubular myopathy; Myotonia congenita; Narcolepsy; Neuro-Behcet's disease; Neuroinflammation; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Neuropathy; Neurosis; Niemann-Pick disease; Non-24-hour sleep-wake disorder; Nonverbal learning disorder; O'Sullivan-McLeod syndrome; Occipital Neuralgia; Occult Spinal Dysraphism Sequence; Ohtahara syndrome; Olivopontocerebellar atrophy; Opsoclonus myodonus syndrome; Optic neuritis; Orthostatic Hypotension; Otosclerosis; Overuse syndrome; Palinopsia; Paresthesia; Parkinson's disease; Paramyotonia congenita; Paraneoplastic diseases; Paroxysmal attacks; Parry-Romberg syndrome; PANDAS; Pelizaeus-Merzbacher disease; Periodic paralyses; Peripheral neuropathy; Pervasive developmental disorders; Phantom limb/Phantom pain; Photic sneeze reflex; Phytanic acid storage disease; Pick's disease; Pinched nerve; Pituitary tumors; PMG; Polyneuropathy; Polio; Polymicrogyria; Polymyositis; Porencephaly; Post-polio syndrome; Postherpetic neuralgia (PHN); Postural hypotension; Prader-Willi syndrome; Primary lateral sclerosis; Prion diseases; Progressive hemifacial atrophy; Progressive multifocal leukoencephalopathy; Progressive supranuclear palsy; Prosopagnosia; Pseudotumor cerebri; Quadrantanopia; Quadriplegia; Rabies; Radiculopathy; Ramsay Hunt syndrome type I; Ramsay Hunt syndrome type II; Ramsay Hunt syndrome type III (e.g., Ramsay-Hunt syndrome); Rasmussen encephalitis; Reflex neurovascular dystrophy; Refsum disease; REM sleep behavior disorder; Repetitive stress injury; Restless legs syndrome; Retrovirus-associated myelopathy; Rett syndrome; Reye's syndrome; Rhythmic Movement Disorder; Romberg syndrome; Saint Vitus dance; Sandhoff disease; Schilder's disease (two distinct conditions); Schizencephaly; Sensory processing disorder; Septo-optic dysplasia; Shaken baby syndrome; Shingles; Shy-Drager syndrome; Sjögren's syndrome; Sleep apnea; Sleeping sickness; Snatiation; Sotos syndrome; Spasticity; Spina bifida; Spinal cord injury; Spinal cord tumors; Spinal muscular atrophy; Spinal and bulbar muscular atrophy; Spinocerebellar ataxia; Split-brain; Steele-Richardson-Olszewski syndrome; Stiff-person syndrome; Stroke; Sturge-Weber syndrome; Stuttering; Subacute sclerosing panencephalitis; Subcortical arteriosclerotic encephalopathy; Superficial siderosis; Sydenham's chorea; Syncope; Synesthesia; Syringomyelia; Tarsal tunnel syndrome; Tardive dyskinesia; Tardive dysphrenia; Tarlov cyst; Tay-Sachs disease; Temporal arteritis; Temporal lobe epilepsy; Tetanus; Tethered spinal cord syndrome; Thomsen disease; Thoracic outlet syndrome; Tic Douloureux; Todd's paralysis; Tourette syndrome; Toxic encephalopathy; Transient ischemic attack; Transmissible spongiform encephalopathies; Transverse myelitis; Traumatic brain injury; Tremor; Trichotillomania; Trigeminal neuralgia; Tropical spastic paraparesis; Trypanosomiasis; Tuberous sclerosis; 22q13 deletion syndrome; Unverricht-Lundborg disease; Vestibular schwannoma (Acoustic neuroma); Von Hippel-Lindau disease (VHL); Viliuisk Encephalomyelitis (VE); Wallenberg's syndrome; West syndrome; Whiplash; Williams syndrome; Wilson's disease; Y-Linked Hearing Impairment; or Zellweger syndrome. In a particular embodiment, the brian disorder is multiple sclerosis.

As used herein, the term “transient receptor potential vanilloid-2 (TRPV2)” refers to a nonspecific cation channel that is a part of the TRP channel family. This channel is composed of six transmembrane spanning regions (S1-S6) with a pore forming loop between S5 and S6. As TRPV2 is a nonspecific cation channel, it is more permeable to calcium ions. The naturally occurring human TRPV2 gene has a nucleotide sequence as shown in Genbank Accession number NM_016113 and the naturally occurring human TRPV2 protein has an aminoacid sequence as shown in Genbank Accession number NP_057197. The murine nucleotide and amino acid sequences have also been described (Genbank Accession numbers NM_011706 and NP035836). In the context of the invention, for the first time, inventors have shown that TRPV2 was abundantly expressed in human brain endothelial cells, notably in endothelial cells of BBB.

As used herein, the term “modulator of TRPV2” refers to an activator or inhibitor of TRPV2. As used herein, the term “activator or inhibitor of TRPV2” refers to a compound that is capable of stimulating or inhibiting the activity and/or expression of TRPV2. As used herein the terms “TRPV2 activity” refers to selectivity filtration, permeability to cations ions such as calcium ions are the activity attributable to TRPV2.

As used herein, the term “activator of TRPV2” refers to a natural or synthetic compound that directly or indirectly increases the TRPV2 activity. It thus refers to any compound able to directly or indirectly increase the transcription, translation, post-translational modification or activity of TRPV2.

As used herein, the term “inhibitor of TRPV2” refers to a natural or synthetic compound that directly or indirectly decreases the TRPV2 activity that has a biological effect to inhibit or significantly reduce the activity and/or expression of TRPV2. It thus refers to any compound able to directly or indirectly decrease the transcription, translation, post-translational modification or activity of TRPV2.

The activator or inhibitor of TRPV2 activity is a small organic molecule, an aptamer an antibody or a polypeptide.

As used herein the term “aptamers” refers to a class of molecule that represents an alternative to antibodies in term of molecular recognition. Aptamers are oligonucleotide or oligopeptide sequences with the capacity to recognize virtually any class of target molecules with high affinity and specificity.

As used herein the term “small organic molecule” refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

In a particular embodiment, the modulator of TRPV2 is an activator of TRPV2. In a particular embodiment, the activator of TRPV2 is cannabidiol (CBD) and its derivatives thereof. Typically CBD is well known in the art, its CAS number is 13956-29-1 and has the following chemical formula and structure in the art: C₂₁H₃₀O₂

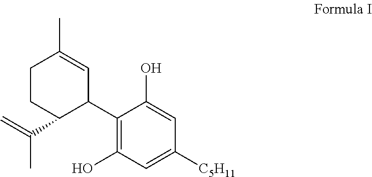

In a particular embodiment, the modulator of TRPV2 is an inhibitor of TRPV2. In a particular embodiment, the inhibitor of TRPV2 is tranilast (TNL) and its derivatives thereof. Typically TNL is well known in the art, its CAS number is 53902-12-8 and has the following structure in the art:

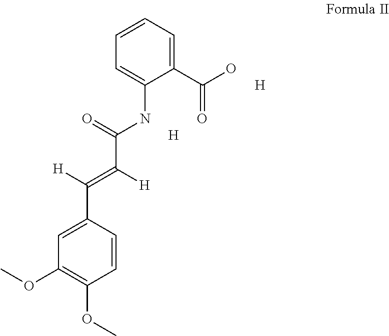

In a further embodiment, the inhibitor of TRPV2 is selected from the following group but not limited to A48, A3, A63, SKF96365, B6, Lumin, as described in Iwata et al 2018, Oncotarget. 2018 Mar. 6; 9(18): 14042-14057.

In another embodiment, the inhibitor of TRPV2 is an antibody.

As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity of TRPV2. Typically, such antibody is suitable to increase the BBB permeability by inhibiting TRPV2.

The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the antibody is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular embodiment, the antibody anti-TRPV2 is conjugated to the drugs. Said antibody is called as antibody drug conjugate (ADC). In a particular embodiment, such antibody is combined with the potency of chemotherapeutic agents. The technology associated with the development of monoclonal antibodies to tumor associated target molecules, the use of more effective cytotoxic agents, and the design of chemical linkers to covalently bind these components, has progressed rapidly in recent years (Ducry L, et a/. Bioconjugate Chemistry, 21:5-13, 2010). In a particular embodiment, the antibody anti-TRPV2 is able to induce cytotoxicity, also known as the antibody-dependent cell-mediated cytotoxicity (ADCC). ADCC is a mechanism of cell-mediated immune defense whereby an effector cell of the immune system actively lyses a target cell, whose membrane-surface antigens have been bound by specific antibodies.

In a particular embodiment, the inhibitor of TRPV2 is an inhibitor of TRPV2 expression. An “inhibitor of TRPV2 expression” refers to a natural or synthetic compound that has a biological effect to inhibit or significantly reduce the expression of the gene encoding for TRPV2. Typically, the inhibitor of TRPV2 expression has a biological effect on one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

In some embodiments, the inhibitor of TRPV2 expression is an antisense oligonucleotide. Anti-sense oligonucleotides, including anti-sense RNA molecules and anti-sense DNA molecules, would act to directly block the translation of TRPV2 mRNA by binding thereto and thus preventing protein translation or increasing mRNA degradation, thus decreasing the level of TRPV2 proteins, and thus activity, in a cell. For example, antisense oligonucleotides of at least about 15 bases and complementary to unique regions of the mRNA transcript sequence encoding TRPV2 can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In a particular embodiment, the inhibitor of TRPV2 expression is a shRNA. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

In some embodiments, the inhibitor of TRPV2 expression is a small inhibitory RNAs (siRNAs). TRPV2 expression can be reduced by contacting the subject or cell with a small double stranded RNA (dsRNA), or a vector or construct causing the production of a small double stranded RNA, such that TRPV2 expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). In a particular embodiment, the siRNA is ALN-PCS02 developed by Alnylam (phase 1 ongoing).

In some embodiments, inhibitor of TRPV2 expression is a ribozyme. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of TRPV2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In some embodiments, the inhibitor of TRPV2 expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

As used herein the terms “administering” or “administration” refer to the act of injecting or otherwise physically delivering a substance as it exists outside the body (e.g., a modulator of TRPV2) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of an anti-TRPV2 antibody for use in a method for modulating blood-brain barrier (BBB) at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic 20 adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Combined Preparation:

The modulator of TRPV2 as described above is combined with classical treatments.

Accordingly, the invention relates to i) a modulator of TRPV2 and ii) a classical treatment used as a combined preparation for modulating the blood brain barrier in a subject.

As used herein, the terms “combined treatment”, “combined therapy” or “therapy combination” refer to a treatment that uses more than one medication. The combined therapy may be dual therapy or bi-therapy.

In a particular embodiment, i) a modulator of TRPV2 and ii) a classical treatment as a combined preparation according to the invention for simultaneous, separate or sequential use in the method for modulating the BBB in a subject.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In a particular embodiment, the classical treatment refers to radiation therapy, immunotherapy or chemotherapy.

In a particular embodiment, the invention relates i) a modulator of TRPV2 and ii) a chemotherapy used as a combined preparation for modulating the blood brain barrier in a subject.

In a particular embodiment, i) a modulator of TRPV2 and ii) chemotherapy as a combined preparation according to the invention for simultaneous, separate or sequential use in the method for modulating the BBB in a subject. Typically, i) CBD and ii) chemotherapy as a combined preparation according to the invention for simultaneous, separate or sequential use in the method for modulating the BBB in a subject.

In a particular embodiment, the invention relates i) a modulator of TRPV2 and ii) chemotherapy used as a combined preparation for increasing the permeability of the BBB in a subject.

In a particular embodiment, i) a modulator of TRPV2 and ii) chemotherapy as a combined preparation according to the invention for simultaneous, separate or sequential use in the method for increasing the permeability of the BBB in a subject.

In a particular embodiment, the invention relates i) a modulator of TRPV2 and ii) a chemotherapy used as a combined preparation for decreasing the permeability of the BBB in a subject.

In a particular embodiment, i) a modulator of TRPV2 and ii) chemotherapy as a combined preparation according to the invention for simultaneous, separate or sequential use in the method for decreasing the permeability of the BBB in a subject.

In a particular embodiment, the invention relates i) a modulator of TRPV2 and ii) a chemotherapy used as a combined preparation for repairing the BBB in a subject.

In a particular embodiment, i) a modulator of TRPV2 and ii) chemotherapy as a combined preparation according to the invention for simultaneous, separate or sequential use in the method for repairing the BBB in a subject.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the invention relates i) a modulator of TRPV2 and ii) a radiotherapy used as a combined preparation for modulating the BBB in a subject.

In a particular embodiment, i) a modulator of TRPV2 and ii) radiotherapy as a combined preparation according to the invention for simultaneous, separate or sequential use in the method for modulating the BBB in a subject.

In a particular embodiment, the invention relates i) a modulator of TRPV2 and ii) a radiotherapy used as a combined preparation for increasing the permeability of the BBB in a subject.

In a particular embodiment, i) a modulator of TRPV2 and ii) radiotherapy as a combined preparation according to the invention for simultaneous, separate or sequential use in the method for increasing the permeability of the BBB in a subject.

In a particular embodiment, the invention relates i) a modulator of TRPV2 and ii) a radiotherapy used as a combined preparation for decreasing the permeability of the BBB in a subject.

In a particular embodiment, i) a modulator of TRPV2 and ii) radiotherapy as a combined preparation according to the invention for simultaneous, separate or sequential use in the method for decreasing the permeability of the BBB in a subject.

In a particular embodiment, the invention relates i) a modulator of TRPV2 and ii) a radiotherapy used as a combined preparation for repairing the BBB in a subject.

In a particular embodiment, i) a modulator of TRPV2 and ii) radiotherapy as a combined preparation according to the invention for simultaneous, separate or sequential use in the method for repairing the BBB in a subject.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the invention relates i) a modulator of TRPV2 and ii) an immune checkpoint inhibitor used as a combined preparation for modulating the blood brain barrier in a subject.

In a particular embodiment, i) a modulator of TRPV2 and ii) an immune checkpoint inhibitor as a combined preparation according to the invention for simultaneous, separate or sequential use in the method for modulating the BBB in a subject.

In a particular embodiment, the invention relates i) a modulator of TRPV2 and ii) an immune checkpoint inhibitor used as a combined preparation for increasing the permeability of the BBB in a subject.

In a particular embodiment, i) a modulator of TRPV2 and ii) an immune checkpoint inhibitor as a combined preparation according to the invention for simultaneous, separate or sequential use in the method for increasing the permeability of the BBB in a subject.

In a particular embodiment, the invention relates i) a modulator of TRPV2 and ii) an immune checkpoint inhibitor used as a combined preparation for decreasing the permeability of the BBB in a subject.

In a particular embodiment, i) a modulator of TRPV2 and ii) an immune checkpoint inhibitor as a combined preparation according to the invention for simultaneous, separate or sequential use in the method for decreasing the permeability of the BBB in a subject.

In a particular embodiment, the invention relates i) a modulator of TRPV2 and ii) an immune checkpoint inhibitor used as a combined preparation for repairing the BBB in a subject.

In a particular embodiment, i) a modulator of TRPV2 and ii) an immune checkpoint inhibitor as a combined preparation according to the invention for simultaneous, separate or sequential use in the method for repairing the BBB in a subject.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-ethyl]amino}-1,2,5-oxadiazole-3 carboximidamide:

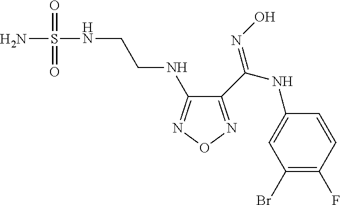 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

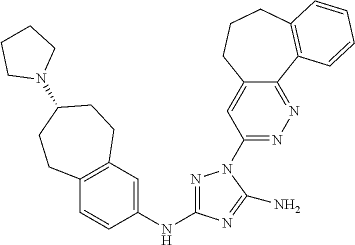 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

Pharmaceutical Composition:

The modulator of TRPV2 for use according to the invention alone and/or combined with classical treatment as described above may be combined with pharmaceutically acceptable excipients, and optionally sustained-release matrices, such as biodegradable polymers, to form pharmaceutical compositions.

Accordingly, in a further aspect, the invention relates to a pharmaceutical composition comprising a modulator of TRPV2 for modulating the BBB.

Accordingly, in a further aspect, the invention relates to a pharmaceutical composition comprising a modulator of TRPV2 for increasing the permeability of the BBB.

Accordingly, in a further aspect, the invention relates to a pharmaceutical composition comprising a modulator of TRPV2 for decreasing the permeability of the BBB.

Accordingly, in a further aspect, the invention relates to a pharmaceutical composition comprising a modulator of TRPV2 for repairing the BBB.

In a particular embodiment, the pharmaceutical composition according the invention, wherein the modulator of TRPV2 is CBD.

In a particular embodiment, the pharmaceutical composition according the invention, wherein the modulator of TRPV2 is TNL.

In a particular embodiment, the pharmaceutical composition according the invention comprising i) a modulator of TRPV2 and ii) a classical treatment.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

Method for Screening:

In a further aspect, the invention relates to a method of screening a drug suitable for the modulating BBB comprising i) providing a test compound and ii) determining the ability of said test compound to activate or inhibit the expression or activity of TRPV2.

Any biological assay well known in the art could be suitable for determining the ability of the test compound to activate or inhibit the activity or expression of TRPV2. In some embodiments, the assay first comprises determining the ability of the test compound to bind to TRPV2. In some embodiments, a population of BBB cells then contacted and activated so as to determine the ability of the test compound to activate or inhibit the activity or expression of TRPV2. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of activating or inhibiting the activity or expression of TRPV2, as determined using in vitro methods described herein, are likely to exhibit similar modulatory capacity in applications in vivo. Typically, the test compound is selected from the group consisting of peptides, petptidomimetics, small organic molecules, antibodies (e.g. intraantibodies), aptamers or nucleic acids. For example the test compound according to the invention may be selected from a library of compounds previously synthesised, or a library of compounds for which the structure is determined in a database, or from a library of compounds that have been synthesised de novo.

The invention will be further illustrated by the following figures and examples. However, these examples and figures should not be interpreted in any way as limiting the scope of the present invention.

### FIGURES

FIG. 1: Expression of TRPV2 in human brain endothelial cells. (a) mRNA levels of TRPV2 were detected by q-RT-PCR in primary cultures of hPBMECs obtained from patients 1 and 2 (see materials and methods) and in hCMEC/D3 cells. Data are expressed as ratio (mean±SEM) of TRPV2 mRNA levels compared with those of the endogenous housekeeping control TBP set at 1. (b) Expression of TRPV2 determined by Western blot of total crude proteins obtained from hCMEC/D3 cells and hPBMECs from patient 3 (see material and methods). β-actin served as a housekeeping control protein.

FIG. 2: Effect of CBD on cell viability. (a) Effects of CBD on hCMEC/D3 cell viability determined by MTT (O.D. 490) in cells treated with different concentrations (0.1, 0.3, 1, 3, 10 μM) of CBD for 24 h. The control group contains the same proportion of CBD vehicle. (b) Concentration-response relationship of CBD on hCMEC/D3 cell viability based on measurements shown in panel (a). (c) Effect of the TNL 50 μM on 3 μM CBD-induced cell viability. (d) The effect of siRNA on the mRNA levels of TRPV2 in hCMEC/D3 cells. Relative mean values of TRPV2 mRNA levels were determined in cells transfected by the negative siRNA (siNEG) or siRNA targeting TRPV2 (siTRPV2) for 72 h. The control group (CTL) was prepared by replacing siRNA by nuclease-free water. (e) A representative experiment of the protein expression of TRPV2 determined by Western blot in hCMEC/D3 cells transfected by siNEG or siTRPV2 at 72 h with densitometric analysis (n=3). (f) Representative time course of [Ca2+]i increase stimulated by 15 μM CBD in cells transfected by siNEG and siTRPV2. (g) The effect of silencing TRPV2 on cell viability. After transfection, cells were re-distributed in 96-well plates at a density of 1×104 cells/well. Cell viability was detected by MTT (O.D.490) after 1, 2, 3 days. (h) The effect of silencing TRPV2 on cell growth. After transfection, cells were re-distributed in 24-well plates at a density of 5×104 cells/well. The number of Trypan blue-stained living cells in each well was counted after 1, 2, 3 days. (i) The effect of silencing TRPV2 in CBD-induced cell viability. Cell viability was determined by MTT after 24 h incubation with 3 μM CBD in cells transfected by siNEG or siTRPV2 (CTL=100%). Data are expressed as mean±SEM. For FIG. 2c, d, and g, inter-group comparisons were performed by ANOVA with Dunnett a posteriori test, NS, not significant, ** p<0. *** p<0.001 versus CTL group, ##, p<0.01 versus 3 μM CBD group, n=3 in duplicate for FIGS. 2d and n=3 with 6 wells per group for FIGS. 2c and g. For FIGS. 2e, f and h, statistical significance was determined by an unpaired t test, NS, not significant, *, p<0.05, *** p<0.001, **** p<0.0001, n=3 in triplicate. For FIG. 2i, statistical significance was determined by an unpaired t test, *, p<0.05, ** p<0.01 versus control group (set at 100%) in corresponding siNEG or siTRPV2 cells, #, p<0.05 versus 3 μM CBD group in siNEG cells, n=6 in triplicate.

FIG. 3: CBD but not GSK1016790A decreased cell viability of hCMEC/D3 cells.

Effect of cannabidiol (CBD) (A) or GSK1016790A (GSK) (B) on cell viability of hCMEC/D3. Cell viability was determined by MTT (O.D. 490) in cells treated with 15 CBD or 1000 μM GSK for 24, 48, and 72 h. Data are expressed as mean±SEM and statistical significance was determined by an unpaired t test, NS, not significant, ***, p<0.001, **** p<0.0001.

FIG. 4: Pharmacological and genetic inhibition of TRPV2 reverse CBD-induced cell death of hCMEC/D3 cells. (A) Effect of the TRPV2 specific antagonist tranilast (TNL) on chronic CBD-induced cell death. hCMEC/D3 cells were incubated with 15 μM CBD for 48 h pre-treated without or with 50 μM tranilast. Cell viability was measured by ATP CellTiter-Glo luminescent cell viability assay. (B) Effect of the TRPV2 specific antagonist tranilast (TNL) on acute CBD-induced cytotoxicity. hCMEC/D3 cells were incubated with 30 μM CBD for 2 h pre-treated without or with 100 μM tranilast. Cell viability was measured by ATP CellTiter-Glo luminescent cell viability assay. (C) Representative time course of the intracellular Ca2+ increase stimulated by 15 μM CBD in cells transfected by the negative siRNA (siNEG) and siRNA TRPV2 (siTRPV2). (D) Effect of silencing TRPV2 in CBD-induced cell cytotoxicity. Cell viability was determined by MTT after 24 h incubation with 15 μM CBD in cells transfected by the negative siRNA (siNEG) or siRNA TRPV2 (siTRPV2). Data are expressed as mean±SEM. For FIGS. 3A and B, inter-group comparisons were performed by ANOVA with Dunnett a posteriori test, NS, not significant, * p<0.05, ** p<0.01, *** p<0.001, **** p<0.0001 compared with CTL group, #, p<0.05 versus 15 μM CBD, ##, p<0.01 versus 30 μM CBD, n=3 in triplicate. For FIGS. 3C and D, statistical significance was determined by an unpaired t test, *, p<0.05, ** p<0.01 versus control group in corresponding siNEG or siTRPV2 cells, ##, p<0.01 versus 3 μM CBD group in siNEG cells, n=6 in triplicate.

### EXAMPLE

Material & Methods

Chemicals and Reagents

Cannabidiol (CBD), ruthenium red (RR), and tranilast (TNL) were all purchased from Sigma (Saint Quentin Fallavier, France). NaCl, NaHCO₃, NaH2PO4, KCl, KH2PO4, CaCl2, and MgSO4 were purchased from Merck (Fontenay sous Bois, France). RNA extraction kits were obtained from Qiagen (Hilden, Germany). Lipofectamine® RNAiMAX transfection reagent, RT-PCR reagents, and primers were obtained from Eurogentec (Liege, Belgium). The Power SYBR Green PCR Master Mix was purchased from Applied Biosystems (Foster City, Calif., USA). All other reagents and chemicals were from Sigma.

Cell Culture Conditions

Human Primary Brain Microvascular Endothelial Cells (hPBMECs). Brain capillary endothelial cells were isolated from surgical resections of patients with brain tumors. The experimentation was conducted in compliance with the French legislation, and the protocol was permitted by the French Ministry of Higher Education and Research (CODECOH DC-2014-2229). In brief, brain capillaries were isolated using soft digestion of patient brain peritumoral tissues and then seeded. Brain primary microvascular endothelial cells were shortly amplified and seeded on Transwell® (Corning) with microporous membranes (pore size: 0.4 μm) in monoculture or in co-culture with the same patient's fresh primary human cultured astrocytes. Cells were cultured in EBM-2 medium (Lonza, Basel, Switzerland) supplemented with 20% serum and growth factors (Sigma).

hCMEC/D3 cells. The hCMEC/D3 human BBB endothelial cell line was kindly given by Doctor Pierre-Olivier COURAUD (Cochin Institute, Paris, France), and was applied for experiments from passages 27 to 33. The growth medium for hCMEC/D3 was EndoGRO complete medium (Merck) supplemented with 1% streptomycin-penicillin (Gibco, Carlsbad, Calif., USA), and 1 ng·mL-1 basic FGF (Sigma) under 5% CO2 and 37° C. The medium contains 5% fetal bovine serum. Plates and flasks were pre-coated with 150 μg·mL-1 rat tail collagen type I (Corning). Every 3-4 days cells were passaged using trypsin/EDTA (Gibco) to detach the cells from the flasks.

HEK-293 cells. HEK293 cells were cultured in Dulbecco's modified Eagle's medium (DMEM) (Gibco) containing 10% fetal bovine serum (Sigma) and 1% streptomycin-penicillin (Gibco) under 5% CO2 and 37° C.

Non-Targeted Proteomic Studies

Reagents

All the reagents used for proteomic studies were of analytical grade. The Protease Inhibitor Cocktail cOmplete® was bought from Sigma. ProteaseMAX surfactant, mass spectrometry grade rLys-C and sequencing grade modified trypsin were acquired from Promega (Charbonnières-les-Bains, France). RIPA buffer was prepared employing analytical grade reagents from Sigma: 50 mmol·L-1 Tris (pH 8.0), 150 mmol·L-1 NaCl, 1% (V/V) Triton X-100, 0.1% (V/V) SDS and 0.5% (W/V) sodium deoxycholate in high purity water. Standard peptides for protein quantification were purchased from Pepscan (Lelystad, The Netherlands).

Protein Extraction and Digestion

hCMEC/D3 cultured cells were washed twice with DPBS buffer. Proteins were extracted using RIPA buffer assisted by ultrasounds in a BioRuptor (Diagenode, Seraing, Belgium). Samples were clarified by centrifugation (10 min at 10,000 g, 4° C.). The amounts of total protein were determined using the MicroBCA® kit from Thermo Scientific (Illkirch, France) according to vendor's procedure. Protein samples were digested as previously reported12. Briefly, denatured and alkylated proteins were cleaned by precipitation using a methanol-chloroform-water. The protein pellet was resuspended using urea and Protease-Max detergent in Tris-HCl buffer (pH 8.5) and digested in tandem using Lys-C and Trypsin endoproteases (enzyme-protein mass ratio=1:50 and 1:100, respectively). Stable isotope labeled (SIL) peptides were added after digestion for absolute quantification. Samples were dried using a centrifugal vacuum concentrator (Maxi-Dry Lyo, Heto Lab Equipment, Denmark), stored at −80° C. and solubilized just before analysis in an aqueous mixture containing 10% acetonitrile plus 0.1% formic acid.

Unlabeled Hi3 Quantification Method

TRPV2 concentration in protein samples from hCMEC/D3 cells was determined using the unlabeled Hi3 quantification method13-15. This method uses a universal response factor which is calculated by the ratio of the absolute concentration of a protein “internal standard” contained in the sample and the sum of response intensity of the three most intense peptides of this internal standard protein, after trypsin hydrolysis of the sample. The internal standard protein selected in this work is the sodium/potassium ATPase subunit alpha-1 pump (ATP1A1) expressed in hCMEC/D3 cells16. In a first step, the concentration of ATP1A1 in the sample was determined by the AQUA method according to the protocol described in previous reports12, 17, 18 using a proteotypic peptide IVEIPFNSTNK (SEQ ID NO:1). In a second step, the sample was analyzed by nanoLC MS/MS in non-targeted mode, which allowed obtaining the sum of response intensity of the three most intense peptides for ATP1A1 and TRPV2.

Multiple Reaction Monitoring (MRM) Assay Development, and Data Analysis

Absolute quantification of ATP1A1 was performed using the absolute quantification of proteins using SIL peptides approach12, 18. Targeted LC-MS/MS analyses were performed on an ACQUITY UPLC H-Class™ System on line with a Waters Xevo™ TQ-S mass spectrometer (Waters, Manchester, UK). Peptides were injected into an ACQUITY UPLC BEH™ C18 column (Peptide BEH™ C18 Column, 300 A, 1.7 μm, 2.1×100 mm; Guyancourt, France) and eluted over a 24 min gradient where the mobile phase consisted in a mixture of water and acetonitrile [formic acid 0.1% (V/V)] with a flow rate of 0.3 mL/min. Eluted molecules underwent positive electrospray ionization with ion spray capillary voltage at 2.80 kV, drying gas flow rate at 1000 L/h, and under a temperature of 650° C. Analysis was performed in MRM mode using three to four transitions per peptide. Skyline (MacLean et al. 2010) software (version 3.1.0.7382) was used for MRM method development and peak integration.

DA Shotgun Proteomics Analysis and Data Treatment

NanoLC-MS/MS untargeted acquisition was performed using a Dionex Ultimate 3000 Rapid Separation LC nano system coupled to a Q-Exactive Plus Orbitrap (Thermo Scientific). The chromatographic solvents were 0.1% (V/V) formic acid in water (A) and 80% acetonitrile, 0.08% formic acid (V/V) (B). Peptides were vacuum-dried, then resuspended in a mixture of 90% water, 10% acetonitrile plus 0.1% trifluoroacetic acid (V/V). The equivalent to 1 mg of peptides was injected into the system and separated on a 50 cm reversed-phase liquid chromatographic column (Pepmap C18; Thermo Scientific) using a gradient of 5% to 40% B in 120 min, followed by 10 min increasing from 40% to 80% B. After 11 min of 80% B (t=131 min), the gradient returned to 5% B to re-equilibrate the column. The mass spectrometer was configured to acquire the MS/MS spectra using a top-10 data-dependent acquisition (DDA). The MS scan range was from 400 to 2000 m/z. Resolution was set to 70 000 for MS scans and 17 500 for MS/MS scans to increase acquisition speed. The MS Automatic Gain Control target was set to 3.106 counts, while MS/MS Automatic Gain Control target was set to 1.105. NanoLC-MS/MS data treatment was performed with Proteome Discoverer v1.4 (Thermo Scientific) using the Mascot search engine (version 2.2.07; Matrix Science) for protein identification against the Human UniProt database (The UniProt Consortium 2014) (release 2016.02, 29 974 entries). Oxidation (Met) was set as variable modification, whereas Carbamidomethylation (Cys) was set as fixed modification. One possible misscleavage was allowed. The enzyme used was trypsin, monoisotopic peptide mass tolerance was set at 10 ppm and fragment mass tolerance was 0.02 Da. Only ions with score superior to 25 were considered. Peptide false discovery rates were calculated from a decoy database using the percolator unction of Proteome Discoverer. Data were filtered to a false discovery rate of 1%.

RNA Isolation and Reverse Transcription

Total RNA was extracted by RNeasy Mini kit (Qiagen) from confluent hCMEC/D3 cells and primary cultures of hPBMECs (patient 1: a 70-years-old female suffering from glioblastoma, peritumoral biopsy; patient 2: a 8-years old boy suffering from cerebellum astrocytoma, peritumoral biopsy). The concentrations and purity of the total RNA samples were determined by spectrophotometry absorption at 260 nm and 280 nm using the NanoDrop□ND-1000 instrument (NanoDrop Technologies, Wilmington, Del., USA). Reverse transcription was achieved using total RNA in reaction mixture system as reported previously19. RT negative controls were obtained by substituting the reverse transcriptase to nuclease-free water in the mixture system. RT incubation condition was shown as follows: 25° C. for 10 min, then at 42° C. for 30 min and at 99° C. for 5 min (PTC-100 programmable thermal controller, MJ research INC, Saint Bruno, Canada, USA). cDNAs were stored at −80° C.

Quantitative Real Time RT-PCR (qRT-PCR)

Gene expression was analysed by SYBR Green fluorescence detection using an ABI Prism 7900 HT Sequence Detection System (Applied Biosystems) as previously reported19. The final reaction mixture system contained diluted Power SYBR Green PCR Master mix kit, cDNA and primers. OLIGO 6.42 software (MedProbe, Lund, Norway) was applied to design primers. The primers for TRPV2 were: forward (5′-3′) CCCGGCTTCACTTCCTCC (SEQ ID NO: 2) and reverse (5′-3′) GCGTCGGTGTTGGCCTGAC (109 bp) (SEQ ID NO: 3). Primers for TBP and ABCB1 were those already described19. RT negative controls and no-template controls showed negligible signals (Ct value >40). Melting curve analysis was used to ensure reaction specificity. cDNAs from HEK-293 cells was used to validate TRPV2 primers. Gene expression was assessed using the Ct value. It was considered un-quantifiable for Ct more than 32 (starting cDNA material was obtained from a 1/80 dilution). The ΔΔCt method was applied to compare TRPV2 mRNA levels in hCMEC/D3 and hPBMEC cells normalised with the housekeeping gene encoding TATA box-binding protein (TBP)19. PCR efficacy was better than 95% for the three genes of interest and results are expressed as fold-change compared to TBP mRNA levels set at 1.

RNA Interference for TRPV2

The negative siRNA (reference 1027284, Neg. siRNA AF 488) was obtained from Qiagen. siRNA for TRPV2 (Silencer® Select Pre-designed siRNA, reference 4392420, ID: 28081) was purchased from Thermo Fisher. The RNA interference experiments were conducted on 6-well plates. Briefly, for TRPV2 siRNA and negative siRNA groups, 20 μM of the TRPV2 siRNA oligonucleotide or the negative control oligonucleotide were diluted in 250 μL of Opti-MEM and 6 μL of RNAiMAX-transfection reagent were diluted in 250 μL of Opti-MEM, pre-incubated for 5 min and then mixed together and incubated for an additional 20 min at room temperature. The control group was prepared by replacing siRNA to nuclease-free water in the Opti-MEM, the mixture only containing 6 μL of RNAiMAX-transfection reagent in 500 μL of Opti-MEM. After the addition of 1 mL of Opti-MEM, the entire mixture was added to the wells and the cells were further cultivated and transfected for an additional 24 h. After 24 h transfection, half of medium was replaced with fresh complete EndoGRO medium and further cultivated for an additional 48 h. The mRNA and protein levels of TRPV2 were analyzed by qRT-PCR and Western-Blot at 72 h as described below, respectively.

Western Blot

Cell lysates of hCMEC/D3 cells and primary cultures of hPBMECs (patient 3: 48-years old, female, gliobastoma, peritumoral biopsy) were obtained with the protein lysis buffer (150 mM NaCl, 50 mM, 0.5% Tris-HCl pH 7.4, 0.5% Triton X100, 0.5% sodium deoxycholate, and protease inhibitor (cOmplete, Sigma). Total proteins were achieved as previously described19. The Bradford assay was applied to quantify protein concentration (BSA as a standard). 60 μg of total proteins were loaded on a 7.5% SDS-polyacrylamide gel and transferred to polyvinylidene difluoride (PVDF) membranes (BioRad, Marne La Coquette, France), and blocked for 2 h with 5% milk. Membranes were then incubated overnight with monoclonal mouse anti-human TRPV2 primary antibody (1/250, sc-390439, Santa Cruz Biotechnology, Dallas, Tex., USA) or monoclonal mouse anti-human β-actin primary antibody (1/3000, Merck-Millipore, Ref: MAB1501R). Anti-mouse IgG conjugated to HRP (1/2000, Santa Cruz Biotechnology) was applied as the secondary antibody for detection using an ECL plus Western Blot Detection System (GE Healthcare, Little Chalfont, UK).

Confocal Immunolocalization

hCMEC/D3 cells were cultured on 8-well ibidi μ-Slide (1.5 polymer coverslip, tissue culture treated, CliniSciences, Nanterre, France). Cells at 80% of confluence were fixed by 3.2% paraformaldehyde containing PBS for 10 min, and permeabilized by 0.2% Triton-X-100 (Sigma) in PBS for 10 min. Following 30 min incubation in blocking solution (0.2% Triton-X-100, 1% BSA and 10% goat serum containing PBS) at room temperature, cells were incubated with rabbit anti-human TRPV2 primary antibody (1:250, ThermoFisher Scientific, Ref: PA1-18346) and rabbit anti human VE-Cadherin primary antibody (1:500, Enzo Life Sciences, Farmingdale, N.Y., USA, Ref: ALX-210-232-C100) overnight at 4° C. After appropriate washing in PBS, the μ-slides were incubated with goat-anti-rabbit-555 (1:500, Santa Cruz Biotechnology) for 2 h at room temperature. Nuclei were stained with Hoechst 33342 (1:10000, ThermoFisher Scientific). Negative control cells were incubated omitting the primary antibodies. Visualization of the proteins was realised under a LEICA TCS SP2 confocal microscope (Oberkochen, Germany).

Intracellular Ca2+ Signal Measurements

Fluorescence measurement of intracellular Ca2+ ([Ca2+]i) concentration was performed in accordance with our optimized protocol as below: hCMEC/D3 cells grown at 100% confluence in 24-well plates were loaded with 2 μM of fluorescent marker, Fluo-4-AM (λex=496 nm, λem=516 nm, F14201, Thermo Fisher Scientific) for 45 min at 37° C. in a loading Hank's buffer (500 μL/well). The cells were washed and replaced with 500 μL/well buffer. After additional 10 min of incubation at 37° C., the 24-well plates were placed into a Victor™ X2 fluorescent heated microplate reader (PerkinElmer, France). When applying antagonists, cells were pre-treated with the compound for 5 min before to start fluorescent signals recording. Data are expressed as F1/F0, where F0 is the average fluorescence of the control group (no agent or no heat stimulation application) and F1 is the actual fluorescence at the corresponding time for the treated group. To visualize clearly and directly the effect of the chemical agonist and antagonist on [Ca2+]i change, hCMEC/D3 cells were seeded in a 8-well ibidi (1.5 polymer coverslip, CliniSciences). Then, the μ-Slide containing 250 μL/well normal buffer was placed on the platform of a ZEISS 515 Roussy confocal microscope (Carl Zeiss), and fluo-4-AM loaded cells were photographed using a time-lapse mode every 60 s during 20 min in a humidified 5% CO2 atmosphere at 37° C. Images of hCMEC/D3 were analysed in Fiji app running Image J software.

Cell Viability Assays

To study the effects of TRPV2 agonists and antagonists and silencing TRPV2 on cell viability, the 3-(4,5-dimethyl-2-thiazolyl)-2,5-diphenyl-2-H-tetrazolium bromide assay (MTT, Sigma-Aldrich) evaluating cell mitochondrial activity and Trypan blue exclusion assay evaluating living cells were applied. Cells were firstly treated under three conditions mentioned above (Control, negative siRNA, and TRPV2 siRNA) in a 6-well plate. After transfection, cells were re-distributed in a new plate with the same cell number and the same medium in each well. Cell viability were analysed at 0, 24 h, 48 h, 72 h after re-distribution. For MTT assay, cells were re-distributed in 96-well plates at a density of 1×104 cells/well, 6 wells per group, one plate four each time. For Trypan blue exclusion assay, cells were re-distributed in 24-well plates at a density of 5×104 cells/well. The number of living cells (not stained by Trypan blue) in each well was counted in a TC20™ Automated Cell Counter (Bio-Rad) at each time point, with 3 wells per group.

To study the effect of CBD on cell viability, hCMEC/D3 cells were firstly plated into 96-well plate at a density of 1×104 cells/well in 200 μL complete culture medium. Cells were seeded and then changed with fresh complete medium containing different concentrations of CBD (0.1, 0.3, 1, 3, 10 μM) or containing the same proportion of CBD vehicle (less than 0.3% methanol) for control group for an additional 24 h incubation (6 wells/group). When studying the possible involvement of TRPV2 in CBD-induced proliferation, cells were pre-treated with 50 μM TNL (TRPV2 specific antagonist) for 5 min before adding CBD in the well. After 24 h treatment, the wells were replaced with 100 μL/well fresh complete medium, and 20 μL MTT solution (diluted in PBS buffer, 5 mg·mL-1) was added to each well. And, the plates were kept at 37° C. for an additional 4 h. Then the medium was removed and replaced with 100 μL DMSO per well, in order to dissolve the formazan. The plates were read using a Victor™ X2 microplate reader at 490 nm (PerkinElmer).

Wound Healing Migration Assay

Cell migration was determined with wound healing assay in hCMEC/D3 as reported previously20. Briefly, a standard wound was created by scratching the cell monolayer of hCMEC/D3 cells with a sterile 200 μL plastic pipette tip and line makers were made at the bottom of plates to indicate the wound edges. After removing cell fragments, the cells were incubated at 37° C. with medium containing 5% FBS. In order to minimize avoid the effect of cell proliferation on would healing assay, the medium was absent of bFGF. The areas of the wound and wound repair activity were photographed by phase contrast microscope (Olympus, Japan) at 0, 4, 8, 24 h. All images were acquired by Histolab software and analysed by Image J. To study the effect of CBD on cell migration, hCMEC/D3 cells were firstly plated into 12-well plate at a density of 1×105 cells/well in 750 μL complete culture medium. After 3 days, cells were prepared for wound healing assay with 100% confluence. When studying the possible involvement of TRPV2 in CBD-induced cell migration, cells were pre-treated with 50 μM TNL for 5 min before adding CBD in the well. To study the effects of silencing TRPV2 channel on cell proliferation, the cells were firstly treated under three conditions mentioned above (Control, negative siRNA, and TRPV2 siRNA) in a 6-wells plate. After transfection, the cells were re-distributed in 12-well plate at a density of 5×105 cells/well in 750 μL complete culture medium. When cells were 100% confluent, the wound healing assay was started following the above-mentioned protocol. Assays were performed 3 times in triplicate.

3D Culture of hCMEC/D3 Human BBB Endothelial Cells in Matrigel

3D culture of hCMEC/D3 human BBB endothelial cells was performed using Matrigel (Corning). Matrigel, stored at 4° C. at least 24 h before the assay, was added to a 48-well plate (150 μL/well), and then the plate was incubated at 37° C. for 1 h to allow Matrigel polymerization. To study the effect of CBD on tube formation, hCMEC/D3 cells were resuspended in fresh complete medium (5×104 cells·mL-1), containing 3 μM CBD or not. 500 μL/well fresh complete medium containing cells were distributed in the 48-well plate. After 2 h, 7 h and 24 h incubation, images of the wells in the plate were taken. When studying the possible involvement of TRPV2 in CBD-induced tube formation, cells were pre-treated with 50 μM TNL for 5 min before adding CBD in the medium. Assays were performed 3 times in triplicate and tubule-like structure lumen count was realized with Image J software.

Establishment of In Vitro Human BBB Model Using hPBMECs

To study the effect of CBD on in vitro human BBB model formation, hPBMECs were isolated from surgical resections of a fourth patient (patient 4: a 50-years-old female suffering from glioma, peritumoral biopsy). hPBMECs were then seeded onto Transwell® inserts with glial cells conditioned medium (50/50). After 24 h co-culture, CBD (1 μM) or the same proportion of vehicle was added into the cell inserts. To study the involvement of TRPV2 in CBD-induced effect, cells were pre-treated with 50 μM TNL for 5 min before adding CBD. The TEER values expressed in Ω·cm2 were recorded after 1, 2, 4, 10, 24, 31, 48, 72, 96, 120 h of CBD treatment.

Statistical Analysis

Data are expressed as mean value±SEM. Statistical analysis was performed using ANOVA with Dunnett a posteriori test to compare different groups with the control. P value<0.05 was considered statistically significant. To calculate EC50 of CBD, the concentration-response data were fitted to a logistic function as follows: Y=Bottom+(Top−Bottom)/(1+10(log EC50−X)); where Y is the response, Y starts at Bottom and goes to Top with a sigmoid shape, X is the log of concentration. To calculate IC50 of antagonists (RR and TNL), the concentration-response data were fitted to a logistic function as follows: Y=100/(1+10(log IC50−X)×HillSlope) where Y is the normalized response from 100% down to 0%, X is the decimal log of concentration, HillSlope is the slope. Data fitting was all performed in GraphPad Prism 5.01 software.

Results

Expression of TRPV2 in Human Brain Endothelial Cells

The concentration of ATP1A1 in hCMEC/D3 protein samples determined by non-targeted proteomic AQUA method was 11.01±0.03 fmol/μg of total proteins, which is consistent with the literature¹⁶. The three most intense peptides for ATP1A1 and TRPV2 as well as the sums of the intensity responses obtained are presented in Table 1.

TABLE 1
Intensity responses of ATPase and TRPV2 in hCMEC/D3
protein samples.

| | | Σ Intensity |
|---|---|---|
| Proteins | Peptides | responses |
| ATPase | GVGIISEGNETVEDIAAR (SEQ ID NO: 4) | 2.54.10⁸ |
| | QGAIVAVTGDGVNDSPALKK (SEQ ID NO: 5) | |
| | IVEIPFNSTNK (SEQ ID NO: 6) | |
| TRPV2 | DGVNACILPLLQIDR (SEQ ID NO: 7) | 1.36.10⁷ |
| | GVPEDLAGLPEYLSK (SEQ ID NO: 8) | |
| | LETLDGGQEDGSEADRGK (SEQ ID NO: 9) | |

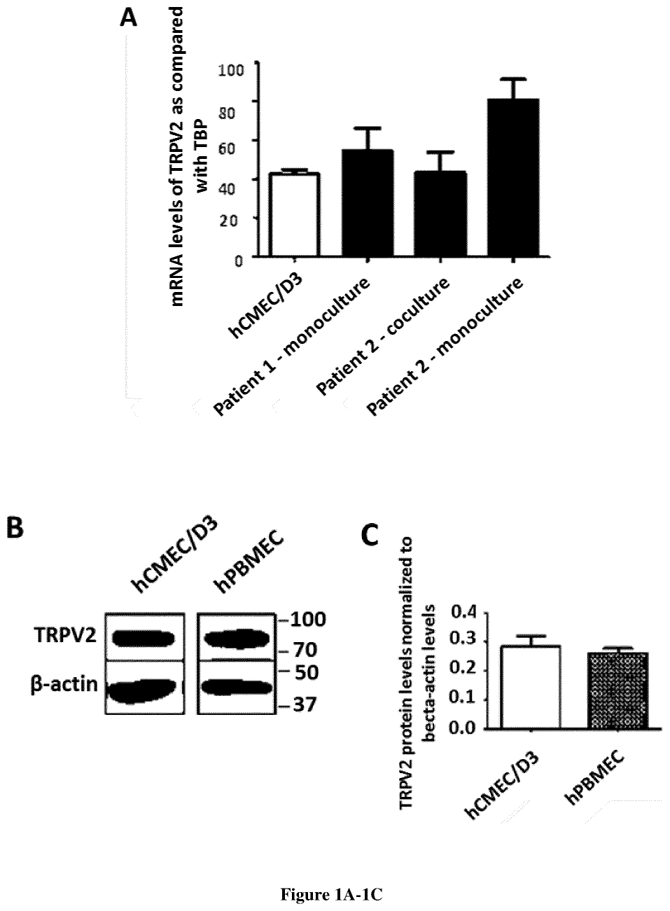

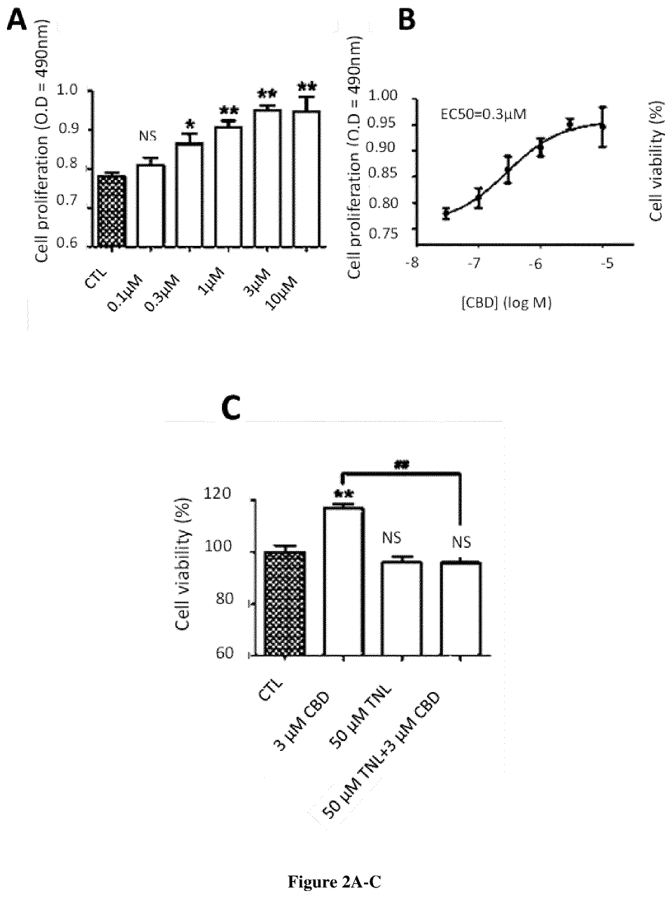

The concentration of TRPV2 calculated by the Hi3 method was thus 0.59 fmol/μg of total proteins, while that of the P-glycoprotein/ABCB1 was barely detected by this method as previously described¹³, suggesting the high abundance of TRPV2 in hCMEC/D3 cells. No other TRPV channels were detected according to MRM assay using targeted LC-MS/MS analyses. Therefore, we focused on the gene and protein expression of TRPV2 in human brain endothelial cells. To assess TRPV2 expression in human brain endothelial cells, we first examined TRPV2 mRNA levels (TBP being normalized at unity) in hCMEC/D3 cells and in primary cultured of hPBMECs from 2 patients with brain tumors. TRPV2 mRNA levels were easily quantifiable with close mRNA levels in both hCMEC/D3 and hPBMECs isolated from patients 1 and 2 (FIG. 1a). TRPV2 mRNA levels were 42- and 12-times higher than those of the TBP and the ABCB1 gene encoding the P-glycoprotein (3.6±0.4, FIG. 1a), a well-known marker of BBB endothelial cells¹⁹, confirming TRPV2 was abundantly expressed in human brain endothelial cells. No significant change was observed for TRPV2 mRNA levels in mono- or co-cultures of hPBMECs with astrocytes from the same adult donor (FIG. 1a, patient 2).

Expression of TRPV2 at protein level was also confirmed by Western blot, where a clear single band was detected at MW (˜90 kDa) from protein samples of hCMEC/D3 cells and hPBMECs of the patient 3 (FIG. 1b), which agreed to the predicted value of TRPV2 (89 kDa). Expression of TRPV2 in hPBMECs (patient 3) was quite similar to that determined in hCMEC/D3 cells (FIG. 1b). Immunofluorescence by microscope confocal analysis revealed an intense staining and a wide distribution of TRPV2 at the plasma membrane and in intracellular compartments with a higher staining in the perinuclear space of hCMEC/D3 cells (data not shown). Negative controls with secondary antibodies incubated without any primary antibody showed no fluorescence signal, indicating the absence of non-specific fluorescence due to secondary antibodies (data not shown). The adherens junction protein, VE-cadherin, was used as a positive control for brain endothelial cells (data not shown)²¹.

Effect of Heat and CBD on Intracellular Ca2+ Levels in hCMEC/D3

TRP channels are known to be activated by heating that increases intracellular Ca2+ levels ([Ca2+]i). We firstly examined the functional responses of hCMEC/D3 cells in terms of [Ca2+]i once exposed to increased temperature. [Ca2+]i increased with temperature over time with a marked increase occurring at around 50° C. (data not shown). This is within the threshold range of temperatures reported to activate TRPV channels 22, particularly TRPV2. However, as this experiment could not discriminate between the relative contribution of different TRP isoforms that might be expressed in hCMEC/D3 cells and activated by heat, we used RR as a non-specific TRPV antagonist and TNL as a potent TRPV2-selective antagonist (data not shown). Ionomycin, a calcium selective ionophore, was used in all further experiments as a positive control able to increase [Ca2+]i levels, assessed using the fluo-4 probe. RR significantly decreased the heat-induced [Ca2+]i signals suggesting the existence of functional TRPVs channels in hCMEC/D3 cells while TNL (50 or 100 μM) significantly decreased heat-induced [Ca2+]i signals as much as RR did (data not shown). The phytocannabinoid CBD, a highly potent agonist of TRPV2, induced a dose-dependent long-lasting increase in [Ca2+]i (data not shown). A slight but significant increase in the [Ca2+]i area under the curve (AUC) over the 20 min incubation was observed from 0.3 μM and reached 1.5-fold at 30 μM CBD as compared to control (data not shown). To visualize cell stimulation by CBD, on-line fluorescent microscopy imaging was also performed using a time-lapse mode every 60 s. As shown, the majority of the cells were stimulated by 15 μM CBD with a lasting elevation of intracellular Ca2+ (data not shown).

Effect of TRP antagonists on CBD-mediated increase in intracellular Ca2+ levels The significant long-lasting elevation of [Ca2+]i levels induced by 15 μM CBD (data not shown) was fully abolished by 10 μM RR pretreatment (data not shown) with an IC50 of 7.7±1.7 μM (data not shown). These results obtained by spectrofluorimetry were also validated by on-line fluorescent microscopy imaging (data not shown). As RR did, TNL (100 μM) fully abolished the CBD-mediated lasting elevation of [Ca2+]i (data not shown), demonstrating a role of TRPV2 in the CBD-mediated elevation of [Ca2+]i. The inhibition effect of TNL was concentration-dependent with an IC50 of 45.9±3.6 μM (data not shown). In addition, on-line recording of [Ca2+]i by fluorescence microscopy in a thermo-regulated chamber showed similar results (data not shown).

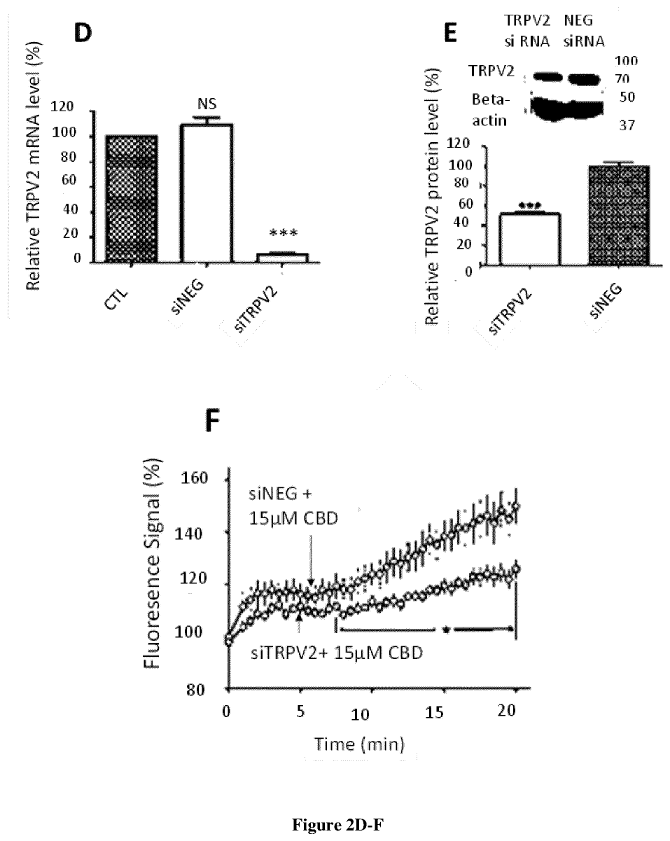

Effect of CBD on Cell Viability in hCMEC/D3 Cells We firstly studied the effect of TRPV2 activation by CBD in the range of 0.3-10 μM for 24 h on hCMEC/D3 cell viability. Compared with control group (containing the same proportion of CBD vehicle), cell viability was not decreased by CBD treatment and on the contrary the MTT absorbance increased suggesting that CBD may induce cell growth. MTT absorbance was significantly increased when treated with CBD from 0.3 μM with a 22.0±1.2% increase at 10 μM (n=6, p<0.01 vs control group) (FIG. 2a) and the CBD effect was dose-dependent with an EC50 about 0.3 μM (FIG. 2b). We checked the effect of CBD on cell proliferation using the Trypan blue exclusion assay. As shown on FIG. 2c, 3 μM CBD increased by 1.2-fold the number of viable cells while 50 μM TNL totally inhibited the CBD effect.

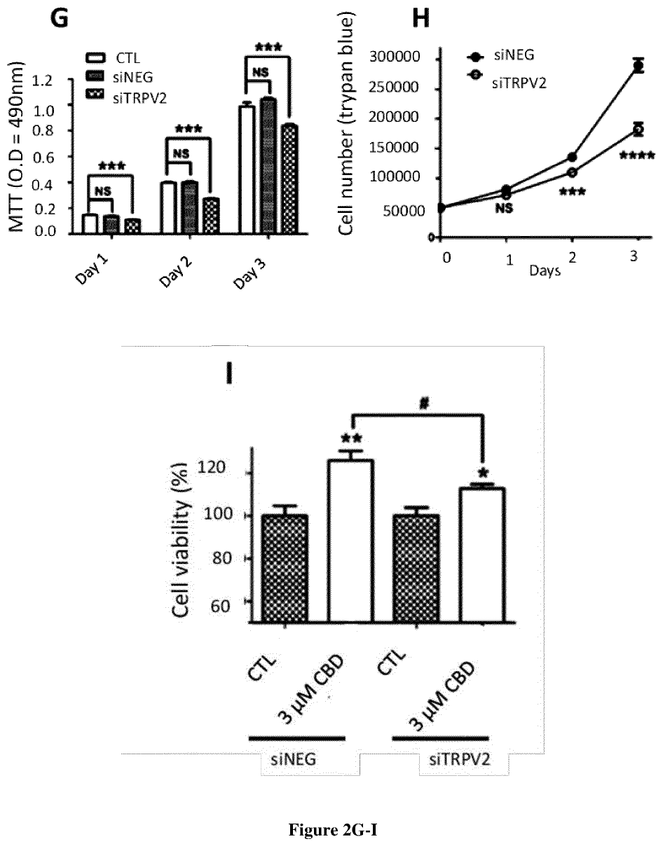

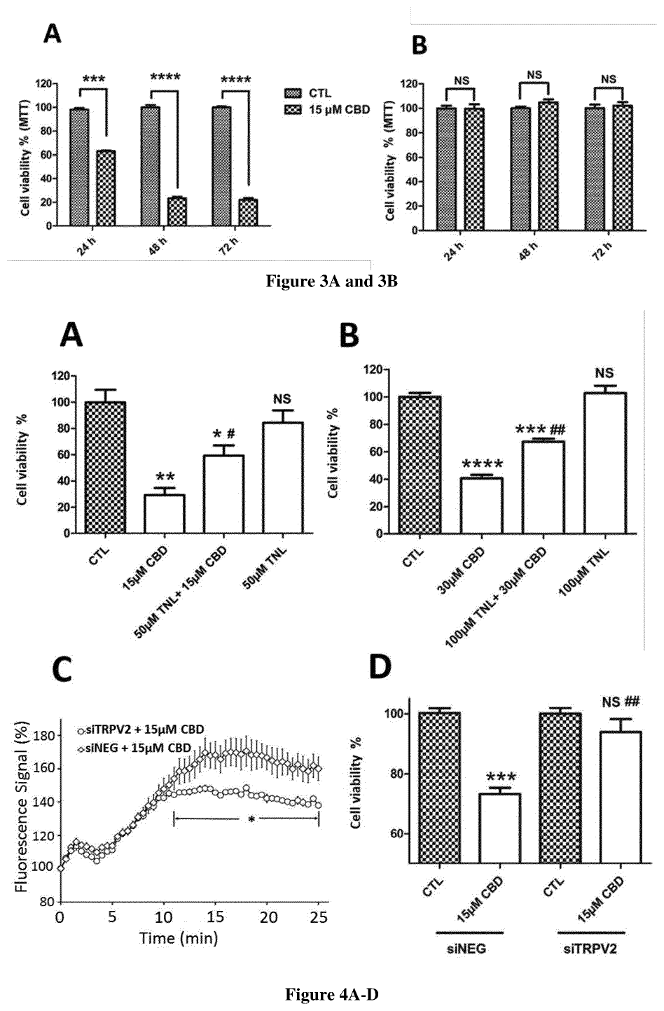

We then focused on 15 μM of TRPV2 and TRPV4, as they were the main functional isoforms of TRPVs expressed in hCMEC/D3 cells and involved in Ca2+ flux. Cell viability determined by MTT assay was significantly decreased by 37%, 77%, and 78% when treated with 15 μM CBD during 24, 48 and 72 h, respectively (FIG. 3A). In contrast, TRPV4 activation by GSK1016790A incubated at 1 μM, a 10-times higher concentration than that producing the maximal effect, did not alter cell viability for 24, 48, or 72 h (FIG. 3B).

To further validate the involvement of TRPV2 in CBD-induced cell death of hCMEC/D3 cells, pharmacological and genetic inhibition strategies were used. The number of viable cells was evaluated by quantitating total ATP in cells treated by CBD. Firstly, hCMEC/D3 cells were exposed for 48 h to CBD (15 μM) to induce cell death with or without TNL. As shown in FIG. 4A, TNL (50 μM) partly reversed the CBD-induced decrease in cell viability (FIG. 4A), suggesting the role of TRPV2 in CBD-induced cell death. We also treated hCMEC/D3 cells with a higher CBD concentration (30 μM) for a 2 h short exposure time to rapidly decrease cell viability. As shown in FIG. 4B, cell viability was significantly decreased upon applying 30 μM CBD for 2 h (FIG. 4B), while this effect was also reversed by co-treatment with 100 μM TNL (FIG. 4B). This further indicates the role of TRPV2 at least partly in the CBD-induced cell death.

To further explore the role of TRPV2 in hCMEC/D3 cell proliferation, siRNA targeting TRPV2 was used to reduce TRPV2 expression. Compared with the control group (no transfection) and the negative group (transfection with siNEG), TRPV2 mRNA levels were significantly reduced by 94% in hCMEC/D3 transfected with TRPV2 siRNA (FIG. 2d, n=3, P<0.001 vs control group), while no difference was observed in cells transfected with the siNEG. FIG. 2e shows also a significant 50% decrease in TRPV2 protein amount assessed by Western blotting in cells transfected with TRPV2 siRNA (FIG. 2e, n=3, p<0.001 vs siNEG). To further examine the effect of TRPV2 silencing on TRPV2 activity, [Ca2+]i were determined in cells transfected by siRNA against TRPV2 or siNEG under CBD stimulation. As shown in FIGS. 2f and 4C, the CBD-mediated increase in [Ca2+]i was significantly reduced in cells silenced for TRPV2 as compared to siNEG cells, especially after 7 min and 10 min treatment with CBD (FIG. 2f, n=3, p<0.05 vs siNEG and FIG. 4C).

We then determined whether or not cell viability and proliferation were altered in cells silenced for TRPV2 using both MTT and Trypan blue exclusion assays. In MTT assays, cells were re-distributed in the 96-well plates with the same cell number in each well (1×104 cells/well) for all 3 groups. Compared with control group, cell proliferation was significantly reduced in cells silenced for TRPV2 by 24.9±1.1%, 31.2±1.3%, 15.1±1.5% at days 1, 2 and 3, respectively, while no significant difference was observed in cells transfected by negative siRNA (FIG. 2g). The effect of TRPV2 siRNA on cell proliferation was also assessed by counting the viable cell number using the Trypan blue assay: cells were re-distributed in 24-well plates with the same cell number in each well (5×104 cells/well) for both siNEG and siTRPV2 groups. The number of viable cells increased from day 0 to day 3 in both siNEG and siTRPV2 cells but it was significantly reduced by 23.0±4.0% and 36.0±3.7% in cells silenced for TRPV2 at day 2 and day 3, respectively (FIG. 2h). Silencing TRPV2 was applied to further validate the involvement of TRPV2 in CBD-induced effect on cell proliferation. Compared with the control group, we still observed a significant CBD-induced proliferation in both cells treated with TRPV2 siRNA (FIG. 2i, p<0.05 vs control group) or negative siRNA (FIG. 2i, p<0.01 vs control group). However, compared with cells treated with negative siRNA, CBD-induced proliferation level was lower in cells treated with TRPV2 siRNA (FIG. 2i, 125.9±4.0% vs 113.0±2.1% for siNEG vs siTRPV2 respectively, p<0.05).

Moreover, a significant decrease in cell viability by 27% after 24 h of 15 μM CBD treatment in cells treated with NEG siRNA (FIG. 4D) was shown. However, compared with the control group, we only observed a slight and not significant cell viability decrease by 6% after 24 h of 15 μM CBD treatment in cells silenced for TRPV2 (FIG. 4D).

These experiments suggested the involvement of TRPV2 in CBD-induced cell death of hCMEC/D3 cells.

Effect of CBD on Cell Migration in hCMEC/D3 Cells

The effect of CBD on cell migration through TRPV2 activation was determined in hCMEC/D3 cells using the wound-healing assay. Cell migration of hCMEC/D3 cells into the acellular area in various control and treated conditions with 3 μM CBD, 50 μM TNL, or 3 μM CBD+50 μM TNL was measured after 4, 8 and 24 h post wound (data not shown). The scratched wound was closed in all groups at 24 h (data not shown). Our result illustrates the mean values of cell migrated area (% of total image area) at 4 h and 8 h. At 4 h, a significant pro-migration effect of 3 μM CBD compared with the control group was already observed that was not significantly decreased by TNL alone. At 8 h, a significant increase (p<0.05) in cell migration was observed in cells treated with 3 μM CBD as compared with control group (data not shown). Co-treatment of CBD with TNL totally inhibited the pro-migration effect of CBD 3 μM (data not shown).

We also determined how cell migration was affected in cells silenced for TRPV2. The images of hCMEC/D3 transfected with siNEG, siTRPV2 cell migration and control group (no transfection), were taken after 4, 8 and 24 h post wound (data not shown). Similar results were observed at 4 h and 8 h. At 8 h, the proportion of migration was significantly higher in control group than in the siNEG group (data not shown) or in the siTRPV2 group (data not shown), but no significant difference was observed between the siNEG group and siTRPV2 group (data not shown). At 24 h, the scratched wound was nearly totally closed in control and siNEG groups, while it remained open in the siTRPV2 group. We observed a significant decreased migration area in the siTRPV2 compared with the siNEG group (data not shown) or control group (data not shown).

Effect of CBD on Tubulogenesis in hCMEC/D3 Cells

As CBD was demonstrated as inducing proliferation and migration of hCMEC/D3 cells, tubulogenesis, a hallmark function of endothelial cells, were also studied to assess whether CBD may have also pro-angiogenic effect. Our result shows that 3 μM CBD induced significantly tube formation at 7 h and 24 h, which was reversed by 50 μM TNL. Compared with the control group, the mean value of closing tube number was significantly increased by 42.0±14.0% by 3 μM CBD at 7 h (data not shown), while this effect was inhibited by co-treatment with 50 μM TNL (data not shown). The results obtained at 24 h were even more pronounced as 3 μM CBD increased the number of closing tubes by 73.0±15.3% compared with control group (data not shown), which could be totally reversed by 50 μM TNL (data not shown).

CBD Increases TEER in hPBMEC Monolayers

We studied the formation of an in vitro human BBB using primary cultures of freshly isolated hPBMECs from a fourth patient. The time course of the TEER was determined for 120 h after cell seeding. TEER values increased upon treatment with 1 μM CBD from post seeding 72 h, while this effect was totally inhibited by co-treatment with 50 μM TNL. One μM CBD has significantly increased TEER by 16.5±2.0% at 120 h post seeding (data not shown), and this increased TEER could be reversed by 50 μM TNL (data not shown).

### REFERENCES

Throughout this application, various references describe the state of the art to which this invention pertains. The disclosures of these references are hereby incorporated by reference into the present disclosure.
1. Sweeney, M. D.; Sagare, A. P.; Zlokovic, B. V. Blood-brain barrier breakdown in Alzheimer disease and other neurodegenerative disorders. Nature reviews. Neurology 2018, 14, (3), 133-150.
2. Ruiz-Valdepenas, L.; Martinez-Orgado, J. A.; Benito, C.; Millan, A.; Tolon, R. M.; Romero, J. Cannabidiol reduces lipopolysaccharide-induced vascular changes and inflammation in the mouse brain: an intravital microscopy study. Journal of neuroinflammation 2011, 8, (1), 5.
3. Mecha, M.; Feliu, A.; Inigo, P. M.; Mestre, L.; Carrillo-Salinas, F. J.; Guaza, C. Cannabidiol provides long-lasting protection against the deleterious effects of inflammation in a viral model of multiple sclerosis: a role for A2A receptors. Neurobiology of disease 2013, 59, 141-50.
4. Pisanti, S.; Malfitano, A. M.; Ciaglia, E.; Lamberti, A.; Ranieri, R.; Cuomo, G.; Abate, M.; Faggiana, G.; Proto, M. C.; Fiore, D.; Laezza, C.; Bifulco, M. Cannabidiol: State of the art and new challenges for therapeutic applications. Pharmacology & therapeutics 2017, 175, 133-150.
5. Pertwee, R. G. Pharmacology of cannabinoid CB1 and CB2 receptors. Pharmacology & therapeutics 1997, 74, (2), 129-80.
6. Qin, N.; Neeper, M. P.; Liu, Y.; Hutchinson, T. L.; Lubin, M. L.; Flores, C. M. TRPV2 is activated by cannabidiol and mediates CGRP release in cultured rat dorsal root ganglion neurons. The Journal of neuroscience: the official journal of the Society for Neuroscience 2008, 28, (24), 6231-8.
7. Kaneko, Y.; Szallasi, A. Transient receptor potential (TRP) channels: a clinical perspective. British journal of pharmacology 2014, 171, (10), 2474-507.
8. De Bock, M.; Wang, N.; Decrock, E.; Bol, M.; Gadicherla, A. K.; Culot, M.; Cecchelli, R.; Bultynck, G.; Leybaert, L. Endothelial calcium dynamics, connexin channels and blood-brain barrier function. Progress in neurobiology 2013, 108, 1-20.
9. Golech, S. A.; McCarron, R. M.; Chen, Y.; Bembry, J.; Lenz, F.; Mechoulam, R.; Shohami, E.; Spatz, M. Human brain endothelium: coexpression and function of vanilloid and endocannabinoid receptors. Brain research. Molecular brain research 2004, 132, (1), 87-92.
10. Hatano, N.; Suzuki, H.; Itoh, Y.; Muraki, K. TRPV4 partially participates in proliferation of human brain capillary endothelial cells. Life sciences 2013, 92, (4-5), 317-24.
11. Moran, M. M. TRP Channels as Potential Drug Targets. Annual review of pharmacology and toxicology 2018, 58, 309-330.
12. Gomez-Zepeda, D.; Taghi, M.; Smirnova, M.; Sergent, P.; Liu, W. Q.; Chhuon, C.; Vidal, M.; Picard, M.; Thioulouse, E.; Broutin, I.; Guerrera, I. C.; Scherrmann, J. M.; Parmentier, Y.; Decleves, X.; Menet, M. C. LC-MS/MS-based quantification of efflux transporter proteins at the BBB. Journal of pharmaceutical and biomedical analysis 2018, 164, 496-508.
13. Gomez-Zepeda, D.; Chaves, C.; Taghi, M.; Sergent, P.; Liu, W. Q.; Chhuon, C.; Vidal, M.; Picard, M.; Thioulouse, E.; Broutin, I.; Guerrera, I. C.; Scherrmann, J. M.; Parmentier, Y.; Decleves, X.; Menet, M. C. Targeted unlabeled multiple reaction monitoring analysis of cell markers for the study of sample heterogeneity in isolated rat brain cortical microvessels. Journal of neurochemistry 2017, 142, (4), 597-609.
14. Silva, J. C.; Gorenstein, M. V.; Li, G. Z.; Vissers, J. P.; Geromanos, S. J. Absolute quantification of proteins by LCMSE: a virtue of parallel MS acquisition. Molecular & cellular proteomics: MCP 2006, 5, (1), 144-56.
15. Wang, H.; Hanash, S. Mass spectrometry based proteomics for absolute quantification of proteins from tumor cells. Methods 2015, 81, 34-40.
16. Ohtsuki, S.; Ikeda, C.; Uchida, Y.; Sakamoto, Y.; Miller, F.; Glacial, F.; Decleves, X.; Scherrmann, J. M.; Couraud, P. O.; Kubo, Y.; Tachikawa, M.; Terasaki, T. Quantitative targeted absolute proteomic analysis of transporters, receptors and junction proteins for validation of human cerebral microvascular endothelial cell line hCMEC/D3 as a human blood-brain barrier model. Molecular pharmaceutics 2013, 10, (1), 289-96.
17. Kamiie, J.; Ohtsuki, S.; Iwase, R.; Ohmine, K.; Katsukura, Y.; Yanai, K.; Sekine, Y.; Uchida, Y.; Ito, S.; Terasaki, T. Quantitative atlas of membrane transporter proteins: development and application of a highly sensitive simultaneous LC/MS/MS method combined with novel in-silico peptide selection criteria. Pharmaceutical research 2008, 25, (6), 1469-83.
18. Kirkpatrick, D. S.; Gerber, S. A.; Gygi, S. P. The absolute quantification strategy: a general procedure for the quantification of proteins and post-translational modifications. Methods 2005, 35, (3), 265-73.
19. Dauchy, S.; Miller, F.; Couraud, P. O.; Weaver, R. J.; Weksler, B.; Romero, I. A.; Scherrmann, J. M.; De Waziers, I.; Decleves, X. Expression and transcriptional regulation of ABC transporters and cytochromes P450 in hCMEC/D3 human cerebral microvascular endothelial cells. Biochemical pharmacology 2009, 77, (5), 897-909.
20. Zarrabeitia, R.; Ojeda-Fernandez, L.; Recio, L.; Bernabeu, C.; Parra, J. A.; Albinana, V.; Botella, L. M. Bazedoxifene, a new orphan drug for the treatment of bleeding in hereditary haemorrhagic telangiectasia. Thrombosis and haemostasis 2016, 115, (6), 1167-77.
21. Weksler, B. B.; Subileau, E. A.; Perriere, N.; Charneau, P.; Holloway, K.; Leveque, M.; Tricoire-Leignel, H.; Nicotra, A.; Bourdoulous, S.; Turowski, P.; Male, D. K.; Roux, F.; Greenwood, J.; Romero, I. A.; Couraud, P. O. Blood-brain barrier-specific properties of a human adult brain endothelial cell line. FASEB journal: official publication of the Federation of American Societies for Experimental Biology 2005, 19, (13), 1872-4.
22. Benham, C. D.; Gunthorpe, M. J.; Davis, J. B. TRPV channels as temperature sensors. Cell calcium 2003, 33, (5-6), 479-87.
23. Earley, S.; Brayden, J. E. Transient receptor potential channels in the vasculature. Physiological reviews 2015, 95, (2), 645-90.
24. Sullivan, M. N.; Francis, M.; Pitts, N. L.; Taylor, M. S.; Earley, S. Optical recording reveals novel properties of GSK1016790A-induced vanilloid transient receptor potential channel TRPV4 activity in primary human endothelial cells. Molecular pharmacology 2012, 82, (3), 464-72.
25. Koles, L.; Garcao, P.; Zadori, Z. S.; Ferreira, S. G.; Pinheiro, B. S.; da Silva-Santos, C. S.; Ledent, C.; Kofalvi, A. Presynaptic TRPV1 vanilloid receptor function is age-but not CB1 cannabinoid receptor-dependent in the rodent forebrain. Brain research bulletin 2013, 97, 126-35.
26. Zhang, Y.; Chen, K.; Sloan, S. A.; Bennett, M. L.; Scholze, A. R.; O'Keeffe, S.; Phatnani, H. P.; Guarnieri, P.; Caneda, C.; Ruderisch, N.; Deng, S.; Liddelow, S. A.; Zhang, C.; Daneman, R.; Maniatis, T.; Barres, B. A.; Wu, J. Q. An RNA-sequencing transcriptome and splicing database of glia, neurons, and vascular cells of the cerebral cortex. The Journal of neuroscience: the official journal of the Society for Neuroscience 2014, 34, (36), 11929-47.
27. Nedungadi, T. P.; Dutta, M.; Bathina, C. S.; Caterina, M. J.; Cunningham, J. T. Expression and distribution of TRPV2 in rat brain. Experimental neurology 2012, 237, (1), 223-37.
28. Che, H.; Yue, J.; Tse, H. F.; Li, G. R. Functional TRPV and TRPM channels in human preadipocytes. Pflugers Archiv: European journal of physiology 2014, 466, (5), 947-59.
29. Che, H.; Xiao, G. S.; Sun, H. Y.; Wang, Y.; Li, G. R. Functional TRPV2 and TRPV4 channels in human cardiac c-kit(+) progenitor cells. Journal of cellular and molecular medicine 2016, 20, (6), 1118-27.
30. Kanzaki, M.; Zhang, Y. Q.; Mashima, H.; Li, L.; Shibata, H.; Kojima, I. Translocation of a calcium-permeable cation channel induced by insulin-like growth factor-I. Nature cell biology 1999, 1, (3), 165-70.
31. Nabissi, M.; Morelli, M. B.; Amantini, C.; Farfariello, V.; Ricci-Vitiani, L.; Caprodossi, S.; Arcella, A.; Santoni, M.; Giangaspero, F.; De Maria, R.; Santoni, G. TRPV2 channel negatively controls glioma cell proliferation and resistance to Fas-induced apoptosis in ERK-dependent manner. Carcinogenesis 2010, 31, (5), 794-803.
32. Liu, Q.; Wang, X. Effect of TRPV2 cation channels on the proliferation, migration and invasion of 5637 bladder cancer cells. Experimental and therapeutic medicine 2013, 6, (5), 1277-1282.
33. Hampson, A. J.; Grimaldi, M.; Axelrod, J.; Wink, D. Cannabidiol and (−)Delta9-tetrahydrocannabinol are neuroprotective antioxidants. Proceedings of the National Academy of Sciences of the United States of America 1998, 95, (14), 8268-73.
34. El-Remessy, A. B.; Al-Shabrawey, M.; Khalifa, Y.; Tsai, N. T.; Caldwell, R. B.; Liou, G. I. Neuroprotective and blood-retinal barrier-preserving effects of cannabidiol in experimental diabetes. The American journal of pathology 2006, 168, (1), 235-44.
35. Iuvone, T.; Esposito, G.; Esposito, R.; Santamaria, R.; Di Rosa, M.; Izzo, A. A. Neuroprotective effect of cannabidiol, a non-psychoactive component from Cannabis sativa, on beta-amyloid-induced toxicity in PC12 cells. Journal of neurochemistry 2004, 89, (1), 134-41.
36. Hind, W. H.; England, T. J.; O'Sullivan, S. E. Cannabidiol protects an in vitro model of the blood-brain barrier from oxygen-glucose deprivation via PPARgamma and 5-HT1A receptors. British journal of pharmacology 2016, 173, (5), 815-25.
37. Nabissi, M.; Morelli, M. B.; Amantini, C.; Liberati, S.; Santoni, M.; Ricci-Vitiani, L.; Pallini, R.; Santoni, G. Cannabidiol stimulates Aml-1a-dependent glial differentiation and inhibits glioma stem-like cells proliferation by inducing autophagy in a TRPV2-dependent manner. International journal of cancer 2015, 137, (8), 1855-69.
38. Massi, P.; Vaccani, A.; Bianchessi, S.; Costa, B.; Macchi, P.; Parolaro, D. The non-psychoactive cannabidiol triggers caspase activation and oxidative stress in human glioma cells. Cellular and molecular life sciences: CMLS 2006, 63, (17), 2057-66.
39. Campos, A. C.; Fogaca, M. V.; Sonego, A. B.; Guimaraes, F. S. Cannabidiol, neuroprotection and neuropsychiatric disorders. Pharmacological research 2016, 112, 119-127.
40. Mecha, M.; Torrao, A. S.; Mestre, L.; Carrillo-Salinas, F. J.; Mechoulam, R.; Guaza, C. Cannabidiol protects oligodendrocyte progenitor cells from inflammation-induced apoptosis by attenuating endoplasmic reticulum stress. Cell death & disease 2012, 3, e331.
41. Koo, C. M.; Kang, H. C. Could Cannabidiol be a Treatment Option for Intractable Childhood and Adolescent Epilepsy? Journal of epilepsy research 2017, 7, (1), 16-20.
42. Geffrey, A. L.; Pollack, S. F.; Bruno, P. L.; Thiele, E. A. Drug-drug interaction between clobazam and cannabidiol in children with refractory epilepsy. Epilepsia 2015, 56, (8), 1246-51.
43. Schmuhl, E.; Ramer, R.; Salamon, A.; Peters, K.; Hinz, B. Increase of mesenchymal stem cell migration by cannabidiol via activation of p42/44 MAPK. Biochemical pharmacology 2014, 87, (3), 489-501.
44. Vaccani, A.; Massi, P.; Colombo, A.; Rubino, T.; Parolaro, D. Cannabidiol inhibits human glioma cell migration through a cannabinoid receptor-independent mechanism. British journal of pharmacology 2005, 144, (8), 1032-6.
45. Solinas, M.; Massi, P.; Cantelmo, A. R.; Cattaneo, M. G.; Cammarota, R.; Bartolini, D.; Cinquina, V.; Valenti, M.; Vicentini, L. M.; Noonan, D. M.; Albini, A.; Parolaro, D. Cannabidiol inhibits angiogenesis by multiple mechanisms. British journal of pharmacology 2012, 167, (6), 1218-31.
46. Nagasawa, M.; Nakagawa, Y.; Tanaka, S.; Kojima, I. Chemotactic peptide fMetLeuPhe induces translocation of the TRPV2 channel in macrophages. Journal of cellular physiology 2007, 210, (3), 692-702.
47. Monet, M.; Lehen'kyi, V.; Gackiere, F.; Firlej, V.; Vandenberghe, M.; Roudbaraki, M.; Gkika, D.; Pourtier, A.; Bidaux, G.; Slomianny, C.; Delcourt, P.; Rassendren, F.; Bergerat, J. P.; Ceraline, J.; Cabon, F.; Humez, S.; Prevarskaya, N. Role of cationic channel TRPV2 in promoting prostate cancer migration and progression to androgen resistance. Cancer research 2010, 70, (3), 1225-35.
48. Kesisis, G.; Broxterman, H.; Giaccone, G. Angiogenesis inhibitors. Drug selectivity and target specificity. Current pharmaceutical design 2007, 13, (27), 2795-809.
49. Patton, A. M.; Kassis, J.; Doong, H.; Kohn, E. C. Calcium as a molecular target in angiogenesis. Current pharmaceutical design 2003, 9, (7), 543-51.
50. Zuccolo, E.; Dragoni, S.; Poletto, V.; Catarsi, P.; Guido, D.; Rappa, A.; Reforgiato, M.; Lodola, F.; Lim, D.; Rosti, V.; Guerra, G.; Moccia, F. Arachidonic acid-evoked Ca(2+) signals promote nitric oxide release and proliferation in human endothelial colony forming cells. Vascular pharmacology 2016, 87, 159-171.
51. Hamdollah Zadeh, M. A.; Glass, C. A.; Magnussen, A.; Hancox, J. C.; Bates, D. O. VEGF-mediated elevated intracellular calcium and angiogenesis in human microvascular endothelial cells in vitro are inhibited by dominant negative TRPC6. Microcirculation 2008, 15, (7), 605-14.
52. Chen, C. K.; Hsu, P. Y.; Wang, T. M.; Miao, Z. F.; Lin, R. T.; Juo, S. H. TRPV4 Activation Contributes Functional Recovery from Ischemic Stroke via Angiogenesis and Neurogenesis. Molecular neurobiology 2018, 55, (5), 4127-4135.
53. Thoppil, R. J.; Cappelli, H. C.; Adapala, R. K.; Kanugula, A. K.; Paruchuri, S.; Thodeti, C. K. TRPV4 channels regulate tumor angiogenesis via modulation of Rho/Rho kinase pathway. Oncotarget 2016, 7, (18), 25849-61.

1. A method for modulating a blood-brain barrier (BBB) in a subject comprising a step of administering to said subject a therapeutically effective amount of a modulator of transient receptor potential vanilloid-2 (TRPV2).

2. The method of claim 1, wherein the modulator of TRPV2 increases blood brain barrier permeability.

3. The method of claim 1, wherein the modulator of TRPV2 decreases blood brain barrier permeability.

4. The method of claim 1, wherein the modulator of TRPV2 repairs the blood brain barrier.

5. The method according to claim 1, wherein the modulator of TRPV2 is an activator of TRPV2.

6. The method according to claim 5, wherein the activator of TRPV2 is cannabidiol (CBD) or a derivative thereof.

7. The method according to claim 1, wherein the modulator of TRPV2 is an inhibitor of TRPV2.

8. The method according to claim 7, wherein the inhibitor of TRPV2 is tranilast (TNL) or a derivative thereof.

9. The method according to claim 7, wherein the inhibitor of TRPV2 is an antibody.

10. The method according to claim 7, wherein the inhibitor of TRPV2 is a small inhibitory RNAs (siRNAs) or an antisense oligonucleotide.

11. The method according to claim 1, wherein the subject has or is susceptible to have Abulia; Agraphia; Alcoholism; Alexia; Alien hand syndrome; Allan-Hemdon-Dudley syndrome; Alternating hemiplegia of childhood; Alzheimer's disease; Amaurosis fugax; Amnesia; Amyotrophic lateral sclerosis (ALS); Aneurysm; Angelman syndrome; Anosognosia; Aphasia; Apraxia; Arachnoiditis; Amold-Chiari malformation; Asomatognosia; Asperger syndrome; Ataxia; Attention deficit hyperactivity disorder; ATR-16 syndrome; Auditory processing disorder; Autism spectrum; Behcets disease; Bipolar disorder; Bell's palsy; Brachial plexus injury; Brain damage; Brain injury; Brain tumor; Brody myopathy; Canavan disease; Capgras delusion; Carpal tunnel syndrome: Causalgia; Central pain syndrome; Central pontine myelinolysis; Centronuclear myopathy; Cephalic disorder; Cerebral aneurysm; Cerebral arteriosclerosis; Cerebral atrophy; Cerebral autosomal dominant arteriopathy with subcortical infarcts and leukoencephalopathy (CADASIL); Cerebral dysgenesis-neuropathy-ichthyosis-keratoderma syndrome (CEDNIK syndrome); Cerebral gigantism; Cerebral palsy; Cerebral vasculitis; Cervical spinal stenosis; Charcot-Marie-Tooth disease; Chiari malformation; Chorea; Chronic fatigue syndrome: Chronic inflammatory demyelinating polyneuropathy (CIDP); Chronic pain; Cockayne syndrome; Coffin-Lowry syndrome; Coma; Complex regional pain syndrome; Compression neuropathy; Congenital facial diplegia; Corticobasal degeneration; Cranial arteritis; Craniosynostosis; Creutzfeldt-Jakob disease; Cumulative trauma disorders; Cushing's syndrome; Cyclothymic disorder; Cyclic Vomiting Syndrome (CVS); Cytomegalic inclusion body disease (CIBD); Cytomegalovirus Infection; Dandy-Walker syndrome; Dawson disease; De Morsier's syndrome; Dejerine-Klumpke palsy; Dejerine-Sottas disease; Delayed sleep phase syndrome; Dementia; Dermatomyositis; Developmental coordination disorder; Diabetic neuropathy; Diffuse sclerosis; Diplopia; Disorders of consciousness; Down syndrome; Dravet syndrome; Duchenne muscular dystrophy; Dysarthria; Dysautonomia; Dyscalculia; Dysgraphia; Dyskinesia; Dyslexia; Dystonia; Empty sella syndrome; Encephalitis; Encephalocele; Encephalotrigeminal angiomatosis; Encopresis; Enuresis; Epilepsy; Epilepsy-intellectual disability in females; Erb's palsy; Erythromelalgia; Essential tremor; Exploding head syndrome; Fabry's disease; Fahr's syndrome; Fainting; Familial spastic paralysis; Febrile seizures; Fisher syndrome; Friedreich's ataxia; Fibromyalgia; Foville's syndrome; Fetal alcohol syndrome; Fragile X syndrome; Fragile X-associated tremor/ataxia syndrome (FXTAS); Gaucher's disease; Generalized epilepsy with febrile seizures plus; Gerstmann's syndrome; Giant cell arteritis; Giant cell inclusion disease; Globoid Cell Leukodystrophy; Gray matter heterotopia; Guillain-Barre syndrome; Generalized anxiety disorder; HTLV-1 associated myelopathy; Hallervorden-Spatz syndrome; Head injury; Headache; Hemifacial Spasm; Hereditary Spastic Paraplegia; Heredopathia atactica polyneuritiformis; Herpes zoster oticus; Herpes zoster Hirayama syndrome; Hirschsprung's disease; Holmes-Adie syndrome; Holoprosencephaly; Huntington's disease; Hydranencephaly; Hydrocephalus; Hypercortisolism; Hypoxia; Ischemic stroke; Immune-Mediated encephalomyelitis; Inclusion body myositis; Incontinentia pigmenti; Infantile Refsum disease; Infantile spasms; Inflammatory myopathy; Intracranial cyst; Intracranial hypertension; Isodicentric 15; Joubert syndrome; Karak syndrome; Kearns-Sayre syndrome; Kinsboume syndrome; Kleine-Levin syndrome; Klippel Feil syndrome; Krabbe disease; Kufor-Rakeb syndrome; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Leukoencephalopathy with vanishing white matter; Lewy body dementia; Lissencephaly; Locked-in syndrome; Lou Gehrig's disease; Lumbar disc disease; Lumbar spinal stenosis; Lyme disease Neurological Sequelae; Machado-Joseph disease (Spinocerebellar ataxia type 3); Macrencephaly; Macropsia; Mal de debarquement; Megalencephalic leukoencephalopathy with subcortical cysts; Megalencephaly; Melkersson-Rosenthal syndrome; Menieres disease; Meningitis; Menkes disease; Metachromatic leukodystrophy; Microcephaly; Micropsia; Migraine; Miller Fisher syndrome; Mini-stroke (transient ischemic attack); Misophonia; Mitochondrial myopathy; Mobius syndrome; Monomelic amyotrophy; Morvan syndrome; Motor Neurone Disease; Motor skills disorder; Moyamoya disease; Mucopolysaccharidoses; Multi-infarct dementia; Multifocal motor neuropathy; Multiple sclerosis; Multiple system atrophy; Muscular dystrophy; Myalgic encephalomyelitis; Myasthenia gravis; Myelinoclastic diffuse sclerosis; Myoclonic Encephalopathy of infants; Myoclonus; Myopathy; Myotubular myopathy; Myotonia congenita; Narcolepsy; Neuro-Behçet's disease; Neuroinflammation; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Neuropathy; Neurosis; Niemann-Pick disease; Non-24-hour sleep-wake disorder; Nonverbal learning disorder; O'Sullivan-McLeod syndrome; Occipital Neuralgia; Occult Spinal Dysraphism Sequence; Ohtahara syndrome; Olivopontocerebellar atrophy; Opsoclonus myodonus syndrome; Optic neuritis; Orthostatic Hypotension; Otosclerosis; Overuse syndrome; Palinopsia; Paresthesia; Parkinson's disease; Paramyotonia congenita; Paraneoplastic diseases; Paroxysmal attacks; Parry-Romberg syndrome; PANDAS; Pelizaeus-Merzbacher disease; Periodic paralyses; Peripheral neuropathy; Pervasive developmental disorders; Phantom limb/Phantom pain; Photic sneeze reflex; Phytanic acid storage disease; Pick's disease; Pinched nerve; Pituitary tumors; PMG; Polyneuropathy; Polio; Polymicrogyria; Polymyositis; Porencephaly; Post-polio syndrome; Postherpetic neuralgia (PHN); Postural hypotension; Prader-Willi syndrome; Primary lateral sclerosis; Prion diseases; Progressive hemifacial atrophy; Progressive multifocal leukoencephalopathy; Progressive supranuclear palsy; Prosopagnosia; Pseudotumor cerebri; Quadrantanopia; Quadriplegia; Rabies; Radiculopathy; Ramsay Hunt syndrome type I; Ramsay Hunt syndrome type II; Ramsay Hunt syndrome type III Rasmussen encephalitis; Reflex neurovascular dystrophy; Refsum disease; REM sleep behavior disorder; Repetitive stress injury; Restless legs syndrome; Retrovirus-associated myelopathy; Rett syndrome; Reye's syndrome; Rhythmic Movement Disorder; Romberg syndrome; Saint Vitus dance; Sandhoff disease; Schilder's disease; Schizencephaly; Sensory processing disorder; Septo-optic dysplasia; Shaken baby syndrome; Shingles; Shy-Drager syndrome; Sjögren's syndrome; Sleep apnea; Sleeping sickness; Snatiation; Sotos syndrome; Spasticity; Spina bifida; Spinal cord injury; Spinal cord tumors; Spinal muscular atrophy; Spinal and bulbar muscular atrophy; Spinocerebellar ataxia; Split-brain; Steele-Richardson-Olszewski syndrome; Stiff-person syndrome; Stroke; Sturge-Weber syndrome; Stuttering; Subacute sclerosing panencephalitis; Subcortical arteriosclerotic encephalopathy; Superficial siderosis; Sydenham's chorea; Syncope; Synesthesia; Syringomyelia; Tarsal tunnel syndrome; Tardive dyskinesia; Tardive dysphrenia; Tarlov cyst; Tay-Sachs disease; Temporal arteritis; Temporal lobe epilepsy; Tetanus; Tethered spinal cord syndrome; Thomsen disease; Thoracic outlet syndrome; Tic Douloureux; Todd's paralysis; Tourette syndrome; Toxic encephalopathy; Transient ischemic attack; Transmissible spongiform encephalopathies; Transverse myelitis; Traumatic brain injury; Tremor; Trichotillomania; Trigeminal neuralgia; Tropical spastic paraparesis; Trypanosomiasis; Tuberous sclerosis; 22q13 deletion syndrome; Unverricht-Lundborg disease; Vestibular schwannoma (Acoustic neuroma); Von Hippel-Lindau disease (VHL); Viliuisk Encephalomyelitis (VE); Wallenberg's syndrome; West syndrome; Whiplash; Williams syndrome; Wilson's disease; Y-Linked Hearing Impairment; or Zellweger syndrome.

12. The method of claim 1, wherein the modulator of TRPV2 is administered in combination with a classical treatment for modulating the blood brain barrier in a subject.

13. The method of claim 12, wherein the classical treatment is radiation therapy, immunotherapy or chemotherapy.

14. A pharmaceutical composition comprising a modulator of TRPV2 for modulating the BBB.

15. A method of screening a drug suitable for modulating the BBB comprising i) providing a test compound and ii) determining the ability of said test compound to activate or inhibit the expression or activity of TRPV2.


SEQUENCE LISTING (9 sequences)

SEQ ID NO: 1 - protein, 11 aa, Artificial
  Synthetic SEQ ID NO 1 proteotypic peptide
IVEIPFNSTNK

SEQ ID NO: 2 - DNA, 18 bp, Artificial
  Synthetic SEQ ID NO 2 forward (5′-3′)
cccggcttcacttcctcc

SEQ ID NO: 3 - DNA, 19 bp, Artificial
  Synthetic SEQ ID NO 3 reverse (5′-3′)
gcgtcggtgttggcctgac

SEQ ID NO: 4 - protein, 18 aa, Artificial
  Synthetic ATPase peptide SEQ ID NO 4
GVGIISEGNETVEDIAAR

SEQ ID NO: 5 - protein, 20 aa, Artificial
  Synthetic ATPase peptide SEQ ID NO 5
QGAIVAVTGDGVNDSPALKK

SEQ ID NO: 6 - protein, 11 aa, Artificial
  Synthetic ATPase peptide SEQ ID NO 6
IVEIPFNSTNK

SEQ ID NO: 7 - protein, 15 aa, Artificial
  Synthetic TRPV2 peptide SEQ ID NO 7
DGVNACILPLLQIDR

SEQ ID NO: 8 - protein, 15 aa, Artificial
  Synthetic TRPV2 peptide SEQ ID NO 8
GVPEDLAGLPEYLSK

SEQ ID NO: 9 - protein, 18 aa, Artificial
  Synthetic TRPV2 peptide SEQ ID NO 9
LETLDGGQEDGSEADRGK